(19) United States
(12) Patent Application Publication
(10) Pub. No.: US 2013/0232852 A1
(43) Pub. Date: Sep. 12, 2013

(54) METHOD FOR TIERED PRODUCTION OF BIOBASED CHEMICALS AND BIOFUELS FROM LIGNIN

(71) Applicants: John R. Peterson, Chardon, OH (US); Vladimir Romakh, Mississauga (CA); Benjamin M.T. Scott, Guelph (CA)

(72) Inventors: John R. Peterson, Chardon, OH (US); Vladimir Romakh, Mississauga (CA); Benjamin M.T. Scott, Guelph (CA)

(73) Assignee: THESIS CHEMISTRY, LLC, Mentor, OH (US)

(21) Appl. No.: 13/453,422

(22) Filed: Apr. 23, 2012

Related U.S. Application Data
(60) Provisional application No. 61/608,936, filed on Mar. 9, 2012.

(51) Int. Cl. C07C 1/20; C07G 1/00; C07C 47/00; C07C 39/00; C07C 35/00; C07C 61/00; C07C 69/74; C10L 1/00; C07C 49/00

(52) U.S. Cl. 44/307; 585/240; 585/469; 530/500; 568/420; 568/303; 568/700; 562/400; 560/1; 568/716; 585/733; 585/638; 585/365

(57) ABSTRACT

The present invention is directed generally to a method of production of value-added, biobased chemicals from lignin sources, including waste lignin. A method of using a depolymerization of lignin to create a tiered production of biobased aromatic chemicals and biofuels is also described herein. The method described herein may also allow for the selective production of the biobased aromatic chemicals and biofuels. Additionally, a reduction of waste products may also be provided from the present method.

This application claims priority to U.S. Ser. No. 61/608,936, entitled A METHOD FOR PRODUCING BIOBASED CHEMICALS FROM PLANT LIGNIN, filed Mar. 9, 2012.

### I. BACKGROUND OF THE INVENTION

A. Field of Invention

The present invention is directed generally to a method of production of value-added, biobased chemicals from lignin sources, including waste lignin. A method of selectively producing biobased aromatic chemicals and biofuels is also described herein.

B. Description of the Related Art

The world currently faces depletion of fossil fuels while demands for these fuels are ever increasing. Petrochemicals provide an energy source and a component of the majority of raw materials used in many industries. In fact, approximately 95% of all chemicals manufactured today are derived from petroleum. However, this heavy reliance upon fossil fuels is creating harm to the environment. The burning of these fossil fuels has led to the pollution of air, water and land, as well as global warming and climate changes. Through the use of fossil fuels, the environment has been harmed, perhaps irreparably, in an effort to meet the nearly insatiable demand for energy and manufactured products. Fossil fuels are a finite natural resource. With the depletion of readily available oil reserves across the globe, the supply chain has shifted to more complex and environmentally risky production technologies. A reduction and conservation of fossil fuels is clearly needed. Some alternatives to fossil fuels, like solar power, wind power, geothermal power, hydropower, and nuclear power, are used to a degree. However, a more efficient use of renewable resources is always being sought.

As a stable and independent alternative to fossil fuels, biomass can be a potentially inexhaustible, domestic, natural resource for the production of energy, transportation fuels, and chemicals. The advantage in use of biomass for such purposes is magnified during an oil crisis, a surge in oil prices, or political unrest within oil producing regions of the world. Biomass includes plant and wood biomass, including agricultural biomass. Biomass can be employed as a sustainable source of energy and is a valuable alternative to fossil fuels. More specifically, the biorefining of biomass into derivative products typically produced from petroleum can help to stop the depletion of petroleum, or at least reduce the current demand and dependence. Biomass can become a key resource for chemical production in much of the world. Biomass, unlike petroleum, is renewable. Biomass can provide sustainable substitutes for petrochemically derived feedstocks used in existing markets.

Biomass is made up primarily of cellulose, hemicellulose, and lignin. These components, if economically separated from one another, can provide vital sources of chemicals normally derived from petrochemicals. The use of biomass can also be beneficial with agricultural and/or woody plants that are sparsely used and plant wastes that currently have little or no use. Biomass can provide valuable chemicals and reduce dependence on coal, gas, and fossil fuels, in addition to boosting local and worldwide economies.

In processes separating biomass, several options are available: the OrganoSolv™ and Alcell® processes which are used to efficiently remove the lignin from the other components under mild conditions, kraft pulping, sulfite pulping, pyrolysis, steam explosion, ammonia fiber explosion, dilute acid hydrolysis, alkaline hydrolysis, alkaline oxidative treatment, and enzymatic treatment. Kraft pulping is by far the dominant chemical pulping practiced in the world. However, often the removal of lignin from plant biomass can be a costly process, and some research efforts are now aimed at designing plants that either deposit less lignin or produce lignins that are more amenable to chemical degradation in order to avoid separating the biomass components.

Although the cellulosic fraction of biomass has garnered substantial attention recently as a feedstock for ethanol biofuel and other basic chemicals, the intrinsic value of the lignin continues to be largely overlooked. Lignin, which can comprise about 15% to about 30% of the organic matrix of woody and agricultural biomass, is the most abundant source of aromatic chemicals outside of crude oil. Lignin can be used in developing technologies that transform plant biomass into value-added aromatic chemicals.

Lignin is a complex, polymeric structure whose exact structure is unknown. This large group of aromatic polymers in lignin can be a result of the oxidative combinatorial linking of the 4-hydroxyphenyl propanoid building blocks provided by nature. The aromatic portion of these building blocks is composed of 4-hydroxyphenyl, guaiacyl (4-hydroxy-3-methoxyphenyl), and syringyl (4-hydroxyl-3,5-dimethoxyphenyl) units. The lignin itself may also vary in the ratio of these units depending on its source.

Because of its make-up, lignin can be a source of aromatic chemicals outside of the conventional sources of petroleum and coal. Lignin may be obtained from wood and/or agricultural sources as fresh biomass. This wood and/or agricultural lignin may be waste lignin or recovered lignin from these sources. Lignin can also be obtained from multiple sources that utilize plant material, including pulp and paper mills and the sugar cane milling industries. It is also a major by-product in the cellulosic biomass-to-ethanol process. Often, these sources of lignin may be considered waste products where there can be an associated cost to dispose of the lignin instead of alternative methods where this lignin can provide value-added materials.

Another source of lignin is the black liquor produced from kraft pulp mills. In the kraft pulping process, lignin-rich black liquor is burnt in a recovery boiler to recover the spent alkali and to generate heat and power for mill operations. Some of the lignin in black liquor could be precipitated and used for value-added applications, especially since a production bottleneck may exist from the thermal capacity of the recovery boiler.

The present invention provides methods for utilizing lignin from biomass and other waste lignin sources, and converting them to value-added biobased materials while minimizing waste products within the process.

### II. SUMMARY OF THE INVENTION

Accordingly, it is an object of the present invention to provide a method for biorefining comprising the steps of providing lignin biomass, processing the lignin biomass, and producing at least one product from the lignin biomass.

According to another embodiment of the invention, the lignin biomass is provided from at least one biomass of plant biomass, woody plant biomass, agricultural plant biomass, and cultivated plant biomass.

According to another embodiment of the invention, the lignin biomass is provided from at least one biomass of fresh plant biomass, recovered plant biomass, pulp and paper mill biomass, cellulosic ethanol refinery biomass, sugarcane mill biomass, and commercial plant biomass fractionator biomass.

According to yet another embodiment of the invention, the lignin biomass is provided from kraft lignin, sulfite pulp mill lignin, soda pulp mill lignin, cellulosic ethanol refinery lignin, and/or commercial plant biomass fractionator lignin.

According to still another embodiment of the invention, the lignin biomass is provided from waste lignin.

One object of the present invention is that the waste lignin is provided by at least one waste lignin from recovered plant biomass waste lignin, kraft pulp mill waste lignin, sulfite pulp mill waste lignin, soda pulp mill waste lignin, cellulosic ethanol refinery waste lignin, commercial plant biomass fractionator waste lignin, and sugar cane mill waste lignin.

Yet another object of the present invention is providing a lignin pretreatment.

Another object of the present invention is the processing of the lignin biomass is provided from at least one process of chemical processing, catalytic processing, biological processing, and pyrolytic processing.

Still another object of the present invention is that the processing of the lignin biomass is provided from at least one process of chemical-induced lignin depolymerization processing, catalytic oxidation processing, catalytic reduction processing, catalytic deoxygenation processing, catalytic reduction/deoxygenation processing, and pyrolytic processing.

Another object of the present invention is that processing of the lignin biomass is provided from at least one process of batch processing or flow processing.

Yet another object of the present invention is that at least one product from the lignin biomass may be used in the tiered production of other chemicals, materials, and products.

Yet another object of the present invention is that at least one product from the lignin biomass comprises at least one chemical of biobased chemicals, biofuels, and lignin residues. Still another object of the present invention is that at least one product from said lignin biomass comprises at least two products of biobased chemicals, biofuels, and lignin residues

Still yet another object of the present invention is that biobased chemicals comprise at least one chemical of commodity chemicals, fine chemicals, and specialty chemicals.

Still yet another object of the present invention is that biobased chemicals comprise at least one product of achiral chemical product, racemic chemical product, and chiral chemical product.

Another object of the present invention is that biobased chemicals comprise at least one chemical of aryl aldehydes, aryl carboxylic acids, aryl esters, aryl ketones, aryl alcohols, and aliphatic carboxylic acids. Still another object of the present invention is that biobased chemicals comprise at least two chemicals of aryl aldehydes, aryl carboxylic acids, aryl esters, aryl ketones, aryl alcohols, and aliphatic carboxylic acids.

Still another object of the present invention is that aryl aldehydes comprise at least one chemical of 4-hydroxybenzaldehyde, vanillin, and syringaldehyde.

Another object of the present invention is that aryl aldehydes comprise at least one chemical of (4-hydroxyphenyl)acetaldehyde, (4-hydroxy-3-methoxyphenyl)acetaldehyde, (4-hydroxy-3,5-dimethoxyphenyl)acetaldehyde, 3-(4-hydroxyphenyl)propionaldehyde, 3-(4-hydroxy-3-methoxyphenyl)propionaldehyde, 3-(4-hydroxy-3,5-dimethoxyphenyl)propionaldehyde, 4-hydroxycinnaminaldehyde, 4-hydroxy-3-methoxycinnaminaldehyde, and 4-hydroxy-3,5-dimethoxycinnaminaldehyde.

Yet another object of the present invention is that aryl carboxylic acids comprise at least one chemical of 4-hydroxybenzoic acid, vanillic acid, and syringic acid.

Still another object of the present invention is that aryl carboxylic acids comprise at least one chemical of (4-hydroxyphenyl)acetic acid, homovanillic acid, homosyringic acid, 3-(4-hydroxyphenyl)propionic acid, 3-(4-hydroxy-3-methoxyphenyl)propionic acid, 3-(4-hydroxy-3,5-dimethoxyphenyl)propionic acid, 4-hydroxycinnamic acid, 4-hydroxy-3-methoxycinnamic acid, and 4-hydroxy-3,5-dimethoxycinnamic acid.

Still yet another object of the present invention is that aryl aldehydes and aryl carboxylic acids comprise at least one chemical of 4-hydroxybenzaldehyde, vanillin, syringaldehyde, 4-hydroxybenzoic acid, vanillic acid, and syringic acid.

One object of the present invention is that aryl esters comprise a C₁-C₁₆ ester of at least one chemical of 4-hydroxybenzoic acid, vanillic acid, syringic acid, (4-hydroxyphenyl)acetic acid, homovanillic acid, homosyringic acid, 3-(4-hydroxyphenyl)propionic acid, 3-(4-hydroxy-3-methoxyphenyl)propionic acid, 3-(4-hydroxy-3,5-dimethoxyphenyl)propionic acid, 4-hydroxycinnamic acid, 4-hydroxy-3-methoxycinnamic acid, and 4-hydroxy-3,5-dimethoxycinnamic acid.

Still yet another object of the present invention is that aryl ketones comprise at least one chemical of 1-(4-hydroxyphenyl)ethanone, 1-(4-hydroxy-3-methoxyphenyl)ethanone, and 1-(4-hydroxy-3,5-dimethoxyphenyl)ethanone.

Another object of the present invention is that aryl ketones comprise at least one chemical of 2-hydroxy-1-(4-hydroxyphenyl)ethanone, 2-hydroxy-1-(4-hydroxy-3-methoxyphenyl)ethanone, 2-hydroxy-1-(4-hydroxy-3,5-dimethoxyphenyl)ethanone, 1-(4-hydroxyphenyl)propanone, 1-(4-hydroxy-3-methoxyphenyl)propanone, 1-(4-hydroxy-3,5-dimethoxyphenyl)propanone, 1-(4-hydroxyphenyl)-2-methyl-1-propanone, 1-(4-hydroxy-3-methoxyphenyl)-2-methyl-1-propanone, 1-(4-hydroxy-3,5-dimethoxyphenyl)-2-methyl-1-propanone, 1-(4-hydroxyphenyl)-2-propanone, 1-(4-hydroxy-3-methoxyphenyl)-2-propanone, and 1-(4-hydroxy-3,5-dimethoxyphenyl)-2-propanone.

Still another object of the present invention is that aryl alcohols comprise at least one chemical of 4-hydroxybenzyl alcohol, 4-hydroxy-3-methoxybenzyl alcohol, 4-hydroxy-3,5-dimethoxybenzyl alcohol, 2-(4-hydroxyphenyl)ethanol, 2-(4-hydroxy-3-methoxyphenyl)ethanol, 2-(4-hydroxy-3,5-dimethoxyphenyl)ethanol, 1-(4-hydroxyphenyl)ethanol, 1-(4-hydroxy-3-methoxyphenyl)ethanol, 1-(4-hydroxy-3,5-dimethoxyphenyl)ethanol, 1-(4-hydroxyphenyl)ethan-1,2-diol, 1-(4-hydroxy-3-methoxyphenyl)ethan-1,2-diol, 1-(4-hydroxy-3,5-dimethoxyphenyl)ethan-1,2-diol, 1-(4-hydroxyphenyl)propanol, 1-(4-hydroxy-3-methoxyphenyl)propanol, 1-(4-hydroxy-3,5-dimethoxyphenyl)propanol, 1-(4-hydroxyphenyl)propan-2-ol, 1-(4-hydroxy-3-methoxyphenyl)propan-2-ol, 1-(4-hydroxy-3,5-dimethoxyphenyl)propan-2-ol, 3-(4-hydroxyphenyl)propan-1-ol, 3-(4-hydroxy-3-methoxyphenyl)propan-1-ol, 3-(4-hydroxy-3,5-dimethoxyphenyl)propan-1-ol, 1-(4-hydroxyphenyl)propan-1,2-diol, 1-(4-hydroxy-3-methoxyphenyl)propan-1,2-diol, 1-(4-hydroxy-3,5-dimethoxyphenyl)propan-1,2-diol, 1-(4-hydroxyphenyl)propan-1,3-diol, 1-(4-hydroxy-3-methoxyphenyl)propan-1,3-diol, 1-(4-hydroxy-3,5-dimethoxyphenyl)propan-1,3-diol, 3-(4-hydroxyphenyl)propan-1,2-diol, 3-(4-hydroxy-3-methoxyphenyl)propan-1,2-diol, 3-(4-hydroxy-3,5-dimethoxyphenyl)propan-1,2-diol, 1-(4-hydroxyphenyl)propan-1,2,3-triol, 1-(4-hydroxy-3-methoxyphenyl)propan-1,2,3-triol, and 1-(4-hydroxy-3,5-dimethoxyphenyl)propan-1,2,3-triol.

Another object of the present invention is that aliphatic carboxylic acids comprise at least one chemical of formic acid, oxalic acid, acetic acid, glycolic acid, glyoxylic acid, propionic acid, lactic acid, and malonic acid.

Yet another object of the present invention is that biobased chemicals comprise at least one chemical of phenols, alkyl phenols, alkenyl phenols, and performance chemicals. Still another object of the present invention is that biobased chemicals comprise at least two chemicals of phenols, alkyl phenols, alkenyl phenols, and performance chemicals.

Still another object of the present invention is that phenols comprise at least one chemical of phenol, guaiacol, and 2,6-dimethoxyphenol.

Still yet another object of the present invention is that alkyl phenols comprise at least one chemical of 4-methylphenol, 3-methylphenol, 2-methylphenol, 4-ethylphenol, 3-ethylphenol, 2-ethylphenol, 4-propylphenol, 3-propylphenol, 2-propylphenol, 4-isopropylphenol, 3-isopropylphenol, 2-isopropylphenol, 4-butylphenol, 3-butylphenol, 2-butylphenol, 4-isobutylphenol, 3-isobutylphenol, 2-isobutylphenol, 4-t-butylphenol, 3-t-butylphenol, 2-t-butylphenol, 2,3-dimethylphenol, 2,4-dimethylphenol, 2,5-dimethylphenol, 2,6-dimethylphenol, 2,3,4-trimethylphenol, 2,4,5-trimethylphenol, and 2,4,6-trimethylphenol.

One object of the present invention is that alkyl phenols comprise at least one chemical of a general molecular structure:

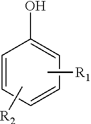

wherein R₁ is selected from among methyl, ethyl, propyl, isopropyl, butyl, isobutyl, and t-butyl; and
wherein R₂ is selected from among ethyl, propyl, isopropyl, butyl, isobutyl, and t-butyl; and
wherein R₁ and R₂ are located at positions 2, 3, 4, or 5 of the phenol ring.

Still yet another object of the present invention is that alkyl phenols comprise at least one chemical of a general molecular structure:

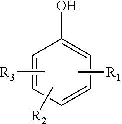

wherein R₁ and R₂ are selected from among methyl, ethyl, propyl, isopropyl, butyl, isobutyl, and t-butyl; and
wherein R₃ is selected from among ethyl, propyl, isopropyl, butyl, isobutyl, and t-butyl; and
wherein R₁, R₂, and R₃ are located at positions 2, 3, 4, or 5 of the phenol ring.

Still another object of the present invention is that alkyl phenols comprise at least one chemical of 2-methoxy-4-methylphenol, 2-methoxy-4-ethylphenol, 2-methoxy-4-propylphenol, 2-methoxy-4-isopropylphenol, 2-methoxy-4-butylphenol, 2-methoxy-4-isobutylphenol, 2-methoxy-4-t-butylphenol, 2,6-dimethoxy-4-methylphenol, 2,6-dimethoxy-4-ethylphenol, 2,6-dimethoxy-4-propylphenol, 2,6-dimethoxy-4-isopropylphenol, 2,6-dimethoxy-4-butylphenol, 2,6-dimethoxy-4-isobutylphenol, and 2,6-dimethoxy-4-t-butylphenol.

Another object of the present invention is that alkenyl phenols comprise at least one chemical of 4-hydroxystyrene, 3-methoxy-4-hydroxystyrene, 3,5-dimethoxy-4-hydroxystyrene, (4-hydroxyphenyl)-1-propene, (4-hydroxyphenyl)-2-propene, eugenol, iso-eugenol, syringeugenol, and iso-syringeugenol.

Still another object of the present invention is that performance chemicals comprise at least one chemical of products comprising phenols, alkyl phenols, and alkenyl phenols.

Another object of the present invention is that biobased chemicals comprise at least one chemical of benzene, toluene, xylenes, mesitylenes, aryl alkanes, aryl alkenes, alkanes, alkenes, cycloalkanes, cycloalkenes, alkyl esters, performance chemicals, and pyrolysis oils. Still another object of the present invention is that biobased chemicals comprise at least two chemicals of benzene, toluene, xylenes, mesitylenes, aryl alkanes, aryl alkenes, alkanes, alkenes, cycloalkanes, cycloalkenes, alkyl esters, performance chemicals, and pyrolysis oils.

Yet another object of the present invention is that biobased chemicals comprise at least one chemical of benzene, toluene, 1,2-dimethylbenzene, 1,3-dimethylbenzene, 1,4-dimethylbenzene, 1,2,3-trimethylbenzene, 1,2,4-trimethylbenzene and 1,3,5-trimethylbenzene.

Still another object of the present invention is aryl alkanes comprise at least one chemical of ethylbenzene, propylbenzene, isopropylbenzene, butylbenzene, isobutylbenzene, and t-butylbenzene.

Still yet another object of the present invention is aryl alkanes comprise at least one chemical of a general molecular structure:

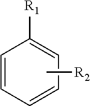

wherein R₁ is selected from among methyl, ethyl, propyl, isopropyl, butyl, isobutyl, and t-butyl; and
wherein R₂ is selected from among ethyl, propyl, isopropyl, butyl, isobutyl, and t-butyl; and
wherein R₂ is located at positions 2, 3, 4, or 5 of the ring.

One object of the present invention is that aryl alkanes comprise at least one chemical of a general molecular structure:

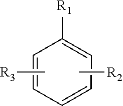

wherein R₁ and R₂ are selected from among methyl, ethyl, propyl, isopropyl, butyl, isobutyl, and t-butyl; and
wherein R₃ is selected from among ethyl, propyl, isopropyl, butyl, isobutyl, and t-butyl; and
wherein R₂ and R₃ are located at positions 2, 3, 4, or 5 of the ring.

Still yet another object of the present invention is that aryl alkenes comprise at least one chemical of styrene, 1-phenyl-1-propene, 1-phenyl-2-propene, 1-(2-methylphenyl)-1-ethene, 1-(3-methylphenyl)-1-ethene, 1-(4-methylphenyl)-1-ethene, 1-(2-methylphenyl)-1-propene, 1-(3-methylphenyl)-1-propene, 1-(4-methylphenyl)-1-propene, 1-(2-methylphenyl)-2-propene, 1-(3-methylphenyl)-2-propene, and 1-(4-methylphenyl)-2-propene.

Still another object of the present invention is that alkanes comprise at least one chemical of hexane, heptane, octane, nonane, 2,3-dimethylheptane, 2,4-dimethylheptane, 2,3,4-trimethylheptane, 2-methyloctane, 3-methyloctane, 4-methyloctane, 2,3-dimethyloctane, 2,4-dimethyloctane, 3,4-dimethyloctane, 2,3,4-trimethyloctane, 2-methylnonane, 3-methylnonane, 4-methylnonane, 5-methylnonane, 2,3-dimethylnonane, 2,4-dimethylnonane, 2,5-dimethylnonane, 3,4-dimethylnonane, 3,5-dimethylnonane, 2,3,4-trimethylnonane, 2,4,5-trimethylnonane, and 3,4,5-trimethylnonane.

Another object of the present invention is that alkenes comprise at least one compound of a partially unsaturated alkane.

Still another object of the present invention is that cycloalkanes comprise at least one chemical of cyclopentane, cyclohexane, cycloheptane, methylcyclopentane, methylcyclohexane, methylcycloheptane, ethylcyclopentane, ethylcyclohexane, ethyl cycloheptane, propylcyclopentane, propylcyclohexane, propylcycloheptane, isopropylcyclopentane, isopropylcyclohexane, isopropylcycloheptane, 1,2-dimethylcyclopentane, 1,3-dimethylcyclopentane, 1,2-dimethylcyclohexane, 1,3-dimethylcyclohexane, 1,4-dimethylcyclohexane, 1,2-dimethylcycloheptane, 1,3-dimethylcycloheptane, and 1,4-dimethylcycloheptane.

Another object of the present invention cycloalkanes comprise at least one chemical of a general molecular structure:

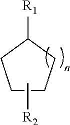

wherein n is 1, 2, or 3; and
wherein R₁ is selected from among methyl, ethyl, propyl, isopropyl, butyl, isobutyl, and t-butyl; and
wherein R₂ is selected from among ethyl, propyl, propyl, isopropyl, butyl, isobutyl, and t-butyl; and
wherein R₂ is located at any ring position other than that of R₁.

Yet another object of the present invention is that cycloalkenes comprise at least one compound of a partially unsaturated cycloalkane.

Still another object of the present invention is that alkyl esters comprise at least one chemical of a general molecular structure:

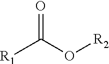

wherein R₁ and R₂ are selected from among methyl, ethyl, propyl, isopropyl, butyl, isobutyl, and t-butyl.

Still yet another object of the present invention is that performance chemicals comprise at least one chemical of benzene, toluene, xylenes, mesitylenes, aryl alkanes, aryl alkenes, alkanes, alkenes, cycloalkanes, cycloalkenes, and alkyl esters.

Still another object of the present invention is that biofuels comprise at least one biobased chemical of alkanes, alkenes, cycloalkanes, cycloalkenes, alkyl esters, benzene, toluene, xylenes, mesitylenes, aryl alkanes, aryl alkenes, alkyl naphthalenes, phenols, alkyl phenols, alkenyl phenols, and pyrolysis oils. Still yet another object of the present invention is that biofuels comprise blends of at least two biobased chemicals of alkanes, alkenes, cycloalkanes, cycloalkenes, alkyl esters, benzene, toluene, xylenes, mesitylenes, aryl alkanes, aryl alkenes, alkyl naphthalenes, phenols, alkyl phenols, alkenyl phenols, and pyrolysis oils.

One object of the present invention is that blends of the biofuels comprise product mixtures of biobased chemicals of similar boiling point range.

One object of the present invention is that blends of the biofuels comprise product mixtures of biobased chemicals with a carbon and hydrogen content of about 80% to about 100%.

Still another object of the present invention is that blends of the biofuels comprise product mixtures of biobased chemicals with a research octane number of at least about 90.

Another object of the present invention is that blends of the biofuels are comprised of at least one fuel of transportation fuels, heating fuels, and fuel additives.

Still another object of the present invention is that transportation fuels serve at least one market of automobile fuels, truck fuels, ship fuels, and aircraft fuels.

Yet another object of the present invention is that heating fuels serve at least one market of home heating fuels, commercial heating fuels, and industrial boiler fuels.

Still another object of the present invention is that fuel additives serve at least one market of transportation fuels and heating fuels.

One object of the present invention is that lignin residue is subjected to further processing to produce at least one additional product.

Another object of the present invention is that lignin residue is further processed from at least one process of chemical processing, catalytic processing, pyrolytic processing, and biological processing.

Still another object of the present invention is that lignin residue is further processed from at least one process of chemical-induced lignin depolymerisation, catalytic oxidation, catalytic reduction, catalytic deoxygenation, catalytic reduction/deoxygenation, and pyrolytic processing.

Another object of the present invention is that lignin residue is further processed from at least one process of batch processing or flow processing.

Still another object of the present invention is that lignin residues comprise at least one product of biobased aromatic chemicals and biobased aromatic fuels.

Yet another object of the present invention is that lignin residues provide energy production.

Still another object of the present invention is that energy production is heat or power.

Still yet another object of the present invention is that lignin biomass has a weight, and a waste product of the lignin biomass is less than 30% of the lignin biomass weight.

Still another object of the present invention is that lignin biomass has a weight, and a waste product of the lignin biomass is less than 20% of the lignin biomass weight.

One object of the present invention is that lignin biomass has a weight, and a waste product of the lignin biomass is less than 10% of the lignin biomass weight.

Still another object of the present invention is that waste products of the processing of the lignin biomass provide energy production.

Another object of the present invention is that energy production is heat or power.

Still another object of the present invention is that the method described herein further comprises the step of recovering and recycling caustic from the processing of the lignin biomass.

Yet another object of the present invention is that size exclusion membrane filtration is used for recovering and recycling caustic from the processing of the lignin biomass.

Still another object of the present invention is that pH precipitation is used for said recovering and recycling caustic from the processing of the lignin biomass.

Still yet another object of the present invention is that the method described herein further comprises the step of functionalizing the lignin biomass prior to producing at least one product from the lignin biomass.

Still another object of the present invention is that the product of the lignin biomass has an economic value higher than boiler fuel.

One object of the present invention is that the processing of the lignin biomass produces at least two products of differing economic value.

One object of the present invention is that the selective production of the product from the lignin biomass occurs.

Still another object of the present invention is the method for biorefining described herein comprising the steps of: providing lignin biomass comprising at least one biomass of plant biomass, woody plant biomass, agricultural plant biomass, cultivated plant biomass, kraft pulping biomass, sulfite pulping biomass, soda pulping biomass, cellulosic ethanol refinery biomass, sugarcane mill biomass, and waste biomass; processing the lignin biomass from at least one process of chemical processing, catalytic processing, biological processing, and pyrolytic processing; processing the lignin biomass from at least one process of chemical-induced lignin depolymerization processing, catalytic oxidation processing, catalytic reduction processing, catalytic deoxygenation processing, catalytic reduction/deoxygenation processing, and pyrolytic processing; functionalizing the lignin biomass prior to producing at least one product from the lignin biomass; producing at least one product from the lignin biomass comprising at least one product of biobased aromatic chemicals, biobased aromatic fuels, and lignin residues; producing a plurality of products from the lignin biomass comprising at least one chemical of aryl aldehydes, aryl carboxylic acids, aryl esters, aryl ketones, aryl alcohols, aliphatic carboxylic acids, phenols, alkyl phenols, alkenyl phenols, performance chemicals, benzene, toluene, xylenes, mesitylenes, aryl alkanes, aryl alkenes, alkanes, alkenes, cycloalkanes, cycloalkenes, alkyl esters, performance chemicals, pyrolysis oils, alkyl naphthalenes, phenols, and alkyl phenols; reducing the waste product of the lignin biomass, wherein the lignin biomass has a weight, and the waste product of the lignin biomass is less than 20% of the lignin biomass weight; producing energy utilizing the lignin residues; producing energy utilizing the waste product of the lignin biomass; and recovering and recycling caustic from the processing of the lignin biomass; wherein selective production of the product of the lignin biomass occurs.

Further, another object of the present invention can be to provide a method for biorefining that is easy to implement and use.

Still other benefits and advantages of the invention will become apparent to those skilled in the art to which it pertains upon a reading and understanding of the following detailed specification.

### III. BRIEF DESCRIPTION OF THE DRAWINGS

The invention may take physical form in certain parts and arrangement of parts, embodiments of which will be described in detail in this specification and illustrated in the accompanying drawings which form a part hereof, and wherein:

FIG. 1 is a flow diagram schematically illustrating lignin sources in the present invention.

FIG. 2 is a flow diagram schematically illustrating the present invention.

FIG. 3 is a flow diagram schematically illustrating another aspect of the present invention.

FIG. 4 is a flow diagram schematically illustrating another aspect of the present invention.

FIG. 5 is a flow diagram schematically illustrating another aspect of the present invention.

FIG. 6 is a flow diagram schematically illustrating another aspect of the present invention.

FIG. 7 is a flow diagram schematically illustrating another aspect of the present invention.

FIG. 8 is a flow diagram schematically illustrating another aspect of the present invention.

FIG. 9 is a flow diagram schematically illustrating another aspect of the present invention.

FIG. 10 is a flow diagram schematically illustrating another aspect of the present invention.

FIG. 11 is a flow diagram schematically illustrating another aspect of the present invention.

### IV. DETAILED DESCRIPTION OF THE INVENTION

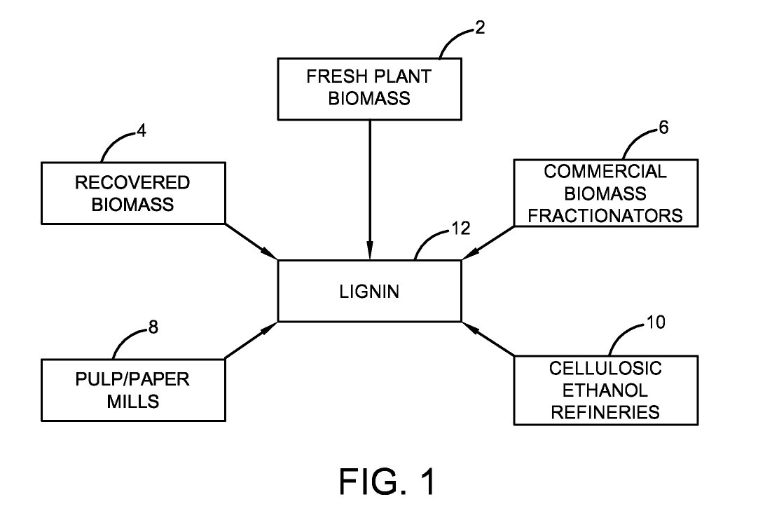

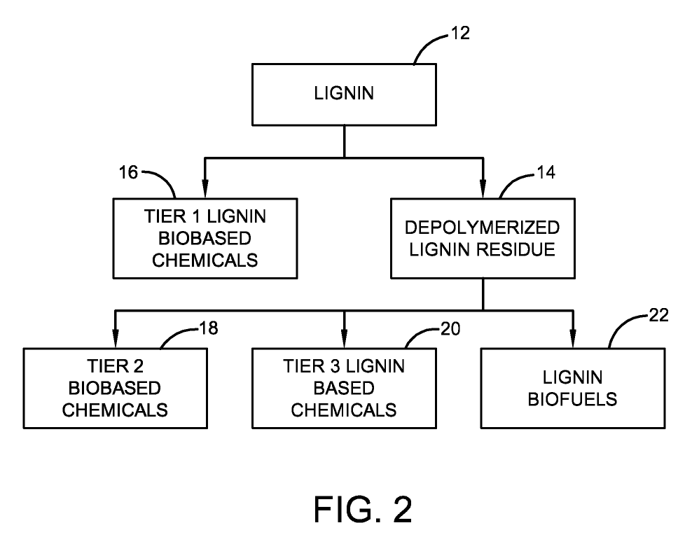

FIG. 1 provides a schematic overview where lignin 12 may be provided from various sources. The sources for the lignin 12 may include fresh plant biomass 2, recovered biomass 4, commercial biomass fractionators 6, pulp and paper mills 8, and/or cellulosic ethanol refineries 10. In processing the lignin 12, it may be converted into other chemical-based products, as shown in FIG. 2.

Lignin 12 may be the most abundant source of aromatic chemicals outside of crude oil and coal. Lignin 12 can be used in developing technologies that transform various sources of biomass and lignin 12 waste into value-added aromatic chemicals. The sources of lignin 12 may include at least one biomass of plant biomass, woody plant biomass, agricultural plant biomass, and cultivated plant biomass. The sources of lignin 12 may include fresh plant biomass 2, recovered biomass 4, commercial biomass fractionators 6, pulp and paper mills 8, and/or cellulosic ethanol refineries 10. Although these sources of lignin 12 can be used, these sources of lignin 12 are not limited to only those listed herein. No matter the origin of the lignin 12, any different sources of lignin 12 may be used within the process described herein.

Lignin 12 can be a structurally complex, polymeric substance made up of 4-hydroxyphenyl propanoid building blocks containing 4-hydroxyphenyl, guaiacyl (4-hydroxy-3-methoxyphenyl), and syringyl (4-hydroxy-3,5-dimethoxyphenyl) units. The abundance of each of these units within the lignin 12 may change somewhat between individual plant species for woody lignin, namely lignin content for hardwoods and softwoods, as well as for agricultural sources and both cultivated and uncultivated plants. This difference in the units based on the species for the lignin 12 can control, or at least predict, the amounts and types of chemical products that may be produced within the process described herein.

To begin the process described herein, fresh plant biomass 2 may be utilized as a lignin source. Fresh plant biomass 2 may be considered to be biomass from agricultural plants, woody plants, and/or other plant biomass sources. Fresh plant biomass 2 may also include cultivated plant biomass. Fresh plant biomass 2 may be used where it may be grown specifically for this application, which may include, but is not limited to, switchgrass, miscanthus, hybrid eucalyptus trees, and hybrid poplar trees. Some fresh plant biomass 2 not specifically grown for this application may include agricultural or tree harvesting surplus. Where fresh plant biomass 2 is used, the lignin 12 can be separated from the other components like cellulose, hemicellulose, and other extractives. After the lignin 12 is separated, it may be added to the process described herein.

Sources of recovered biomass 4 may include several biomass waste products. The recovered biomass 4 can include wood chips, sawdust, recovered wood, wheat straw, corn stover and other agricultural products typically left to rot in the field, and lawn and tree maintenance byproducts. Another potential source of lignin 12 from recovered biomass 4 may include sugar cane milling. Sugar cane milling may provide lignin 12 since bagasse, or sugarcane waste fiber, can be generated. Bagasse is the name given to the discarded husks of the sugarcane plant after they have been pressed to extract the juices which are refined to make sugar. This agricultural waste can be very plentiful and may otherwise be burnt or discarded in the sugar cane milling process. Recovered biomass 4 may also include other waste products, including at least one waste lignin of sulfite pulping mill waste lignin, kraft pulping mill waste lignin, soda pulping mill waste lignin, cellulosic ethanol refinery waste lignin, and sugar cane mill waste lignin.

Both the fresh plant biomass 2 and the recovered biomass 4 may be treated to provide lignin 12 using any of the methods described in U.S. utility applications: A METHOD FOR PRODUCING BIOBASED CHEMICALS FROM PLANT BIOMASS (U.S. application Ser. No. 13/292,222 filed Nov. 9, 2011), A METHOD FOR PRODUCING BIOBASED CHEMICALS FROM WOODY BIOMASS (U.S. application Ser. No. 13/292,437 filed Nov. 9, 2011), A METHOD FOR PRODUCING BIOBASED CHEMICALS FROM AGRICULTURAL BIOMASS (U.S. application Ser. No. 13/292,531 filed Nov. 9, 2011), and A METHOD FOR PRODUCING BIOBASED CHEMICALS FROM CULTIVATED PLANT BIOMASS (U.S. application Ser. No. 13/292,632 filed Nov. 9, 2011).

Another source of lignin 12 may be commercial biomass fractionators 6. These commercial biomass fractionators 6 can be a thermal and/or mechanical processor which directly inputs raw biomass such as fresh plant biomass 2, woodchips and crop waste and produces multiple component streams, which may include sugars, cellulose, hemicellulose, and lignin 12. Some of these component streams may include lignin 12 streams to produce useful products such as benzene, toluene, and/or xylene (often referred to as “BTX”). Within the process, the biomass may be treated to yield a highly pure cellulose fraction. Several different methods may be used for the separation, including pH, temperature, and pressure adjustments. A reaction involving enzymes may also be used. Other methods of fractionation may include chemical, mechanical, and biological methods. For instance, the biomass fractionator may separate the cellulose out by hot water treatments, hot alkaline treatments, and/or an alkaline oxidation step. Although the commercial biomass fractionators 6 may provide useful biobased products, they may also produce or leave behind other solids comprising of lignin 12. Instead of becoming a waste product, these lignin 12 solids may be used within the process described herein.

Pulp and paper mills 8 may also contribute to the lignin 12 from kraft pulping, sulfite pulping, and soda pulping. Lignin 12 can be removed during paper processing in a pulp and paper mills 8, where it is typically viewed as an undesirable component of biomass that requires both energy and chemicals to remove it during the pulping operation. These pulp and paper mills 8 may generally recover the lignin 12 as a by-product of the pulping process and may use it as boiler fuel. This removal of lignin 12 may be done by a chemical removal, with or without mechanical means. Some chemical methods of lignin 12 removal from pulp and paper mills 8 may be kraft pulping, sulfite pulping, and soda pulping.

The more dominant chemical pulping technique employed can be kraft processing, which employs high pHs by using considerable amounts of aqueous sodium hydroxide and sodium sulfide at high temperatures to degrade cellulosic biomass into cellulose, hemicellulose, and lignin 12 in a stepwise process. In the kraft process, black liquor can be burnt in a recovery boiler to recover the spent alkali and to generate heat and power for mill operations. However, some of the lignin 12 in black liquor can be precipitated and used for value-added applications where these exist. This conversion to value-added applications may be particularly attractive for a kraft pulping mill where a production bottleneck exists due to the thermal capacity of the recovery boiler. This process may provide kraft lignin.

The sulfite processing yielding lignosulfonates can also be relatively common in the pulp and paper industry. The sulfite process may be conducted between about pH 2 to about pH 12 using sulfite with a counterion. This counterion may be either calcium or magnesium. The product may be soluble in water as well as some highly polar organics and amines.

The soda pulp mill may also provide another chemical pulping process where caustic soda can be used to produce pulp. Although it is an old method, it can be effective in separating pulp from wood.

Another source of lignin 12 may also be cellulosic ethanol refineries 10. With the cellulosic ethanol refineries 10, they may produce lignin 12 and other by-products in the cellulosic biomass-to-ethanol process, which can also be used to produce energy required for the ethanol production process. Cellulosic ethanol refineries 10 produce ethanol fuel. The cellulosic ethanol can be made from plant materials like switchgrass, wheat stalks, corn stover, and woody biomass.

Cellulosic ethanol refineries 10 may use the Organosolv process or the Alcell process to obtain lignin 12. Organosolv lignin may be obtained by treatment of fresh plant biomass 2 or bagasse, the fibrous residue that remains after plant material may be treated with various organic solvents. The Organosolv process may produce separate streams of cellulose, hemicelluloses, and lignin 12. It can be considered environmentally friendly because it may not use the sulfides, sulfites, and harsh conditions used in the kraft or lignosulfonate pulping processes, but it can have a higher cost because of the solvent recovery in this process. Some processes that may be used to separate the biomass to obtain lignin 12 can include any of the methods described in U.S. utility applications: A METHOD FOR PRODUCING BIOBASED CHEMICALS FROM PLANT BIOMASS (U.S. application Ser. No. 13/292,222 filed Nov. 9, 2011), A METHOD FOR PRODUCING BIOBASED CHEMICALS FROM WOODY BIOMASS (U.S. application Ser. No. 13/292,437 filed Nov. 9, 2011), A METHOD FOR PRODUCING BIOBASED CHEMICALS FROM AGRICULTURAL BIOMASS (U.S. application Ser. No. 13/292,531 filed Nov. 9, 2011), and A METHOD FOR PRODUCING BIOBASED CHEMICALS FROM CULTIVATED PLANT BIOMASS (U.S. application Ser. No. 13/292,632 filed Nov. 9, 2011). Another process to obtain lignin 12 that may be used at cellulosic ethanol refineries 10 may include acidic hydrolysis and/or enzymatic reactions. Typically, the lignin 12 recovered from the cellulosic ethanol refineries 10 may be used as boiler fuel. Additionally, the lignin 12 recovered from the cellulosic ethanol refineries 10 may undergo a pretreatment prior to entry into the process described herein. The purpose of this lignin pretreatment may be to remove unwanted impurities from the lignin 12 and may include a series of steps to further separate lignin 12 from the other components of biomass such as cellulose and hemicellulose as well as small amounts of fats, oils, resins, pitches, waxes, other extractables present in the biomass, or the salts, enzymes, and cellular debris contaminating the lignin from biomass processing. A lignin pretreatment process is described in detail in A METHOD FOR PRODUCING BIOBASED CHEMICALS FROM PLANT BIOMASS (U.S. application Ser. No. 13/292,222 filed Nov. 9, 2011).

Although several sources for lignin are presented herein, those sources for lignin are not limited to those listed. Any lignin 12 provided may be used within the process described to create value-added product(s). Producing these chemicals may provide a reduction in the costs associated with waste disposal of lignin 12 and a means to generate income from biobased chemical production. Besides waste product sources of lignin 12 for the recovered biomass 4, lignin 12 waste from the lignin 12 processing may also provide a source for producing energy. This waste may include recovered plant biomass, kraft pulp mill waste lignin, sulfite pulp mill waste lignin, soda pulp mill waste lignin, cellulosic ethanol refinery waste lignin, commercial plant biomass fractionator waste lignin, and sugar cane mill waste lignin. In this reduction of waste for the process described herein, the waste product of the lignin biomass may be less than 30% of the lignin weight. It may also be less than 20% of the lignin weight. It may also be less than 10% of the lignin weight. These waste products, although reduced, may be converted into producing energy which utilizes the waste product, providing value to the process. This energy production may be heat and/or power.

FIG. 2 shows an overview of the process described herein. This overview of the tiered biobased chemical and/or biofuel production from lignin 12 may allow for selective production of certain chemicals. Lignin 12 may be converted into tier 1 lignin biobased chemicals 16 and/or depolymerized lignin residue 14. If the lignin 12 is converted into depolymerized lignin residue 14, then tier 2 lignin biobased chemicals 18, tier 3 lignin biobased chemicals 20, and/or lignin biofuels 22 may be formed. This conversion may provide a selective production of said product of said lignin biomass occurs.

Within the process(es) described in FIG. 2, the lignin 12 may undergo chemical processing, catalytic processing, pyrolytic processing, and/or biological processing in the selective production of chemicals and biofuels. There may be more than one of these process treatments used for the production of biobased end-products. Additionally, any of these process treatments may be repeated to provide the requirements for the processing to biobased products. For the various processes used for the lignin 12, batch and/or flow processing may be used.

Some of the chemical processing may include reactions with water at elevated temperatures, depolymerization, base hydrolysis, reduction, deoxygenation and reduction/deoxygenation, and/or oxidation and decarboxylation processes. Many of the chemical processing methods may provide an economic conversion of lignin 12 into other chemicals like into tier 1 lignin biobased chemicals 16, tier 2 lignin biobased chemicals 18, tier 3 lignin biobased chemicals 20, and/or lignin biofuels 22. These chemicals may include commodity chemicals, fine chemicals, and specialty chemicals. These chemicals may also include achiral products, racemic products, and chiral products.

For the lignin 12 biomass, the catalytic processing may help to break down the lignin 12. Lignin 12 comprises a complex, polymeric network of 4-hydroxyphenyl propanoid building blocks. These building blocks may be ring-like monomeric nine carbon hydrocarbon units that are principally interconnected by C—O bonds and C—C bonds. Cleaving these bonds without breaking open the individual ring structures may produce useful chemical building blocks, rather than a mixture of short chain hydrocarbons. The catalyst used in the catalytic processing may consist of a metal catalyst. In cleaving these bonds, metal catalysts may contain, but are not limited to, salts and complexes of vanadium, niobium, chromium, molybdenum, tungsten, manganese, rhenium, iron, ruthenium, osmium, cobalt, rhodium, iridium, nickel, palladium, platinum, copper, or combinations thereof. Also, the catalyst selected may provide specific cleaving of the bonds in order to produce desired biobased chemicals.

For the lignin 12 biomass the biological processing may occur through a number of different methods. These methods for biological processing may use, but are not limited to, enzyme, and/or microorganism (i.e., bacteria, fungi, etc.) degradation, or some combination thereof with chemical degradation.

For the lignin 12 biomass, the pyrolytic processing may also occur. Pyrolytic processing may provide a range of products from the current state of the art: “fast pyrolysis” (or sometimes called “flash pyrolysis”). Pyrolytic processing may include thermolysis, which is pyrolysis in the absence of air, and hydrogenolysis, which is pyrolysis in the presence of a hydrogen source. However, pyrolytic processing may not always be economically attractive.

From the various types of lignin 12 processing, including at least one processing step of chemical processing, catalytic processing, biological processing, and pyrolytic processing, biobased chemicals from the various tiers as well as lignin biofuels 22 may be produced. Specifically, the lignin 12, in its conversion, may also undergo chemical-induced lignin depolymerization processing, catalytic oxidation processing, catalytic reduction processing, catalytic deoxygenation processing, catalytic reduction/deoxygenation processing, and/or pyrolytic processing.

With respect to the chemical processing in FIG. 2, lignin 12 may be reacted under selective, mild reaction conditions to produce the highest value tier 1 lignin biobased chemicals 16. The depolymerization of lignin 12 can occur under caustic conditions, therefore solubilizing the lignin 12 and producing the tier 1 lignin biobased chemicals 16. This process may be referred to as “Base-Induced Depolymerization.” The process may also be referred to as “Base-Catalyzed Depolymerization;” however, lignin is a highly complex biopolymer and this process may or may not be truly catalytic in base. Tier 1 lignin biobased chemicals 16 may be purified chemical substances.

A by-product of the reaction from lignin 12 may be a caustic solution of depolymerized lignin 14. The caustic solution of depolymerized lignin 14 may then be transformed under catalytic processes to tier 2 lignin biobased chemicals 18, tier 3 lignin biobased chemicals 20, and/or lignin biofuels 22. Tier 2 lignin biobased chemicals 18 and tier 3 lignin biobased chemicals 20 may be purified chemical substances. Tier 2 lignin biobased chemicals 18 and tier 3 lignin biobased chemicals 20 may also be performance product mixtures. Biofuels 22 can be blends of compounds of a desired boiling point range, preferred carbon and hydrogen content, low sulfur content, and appropriate octane number.

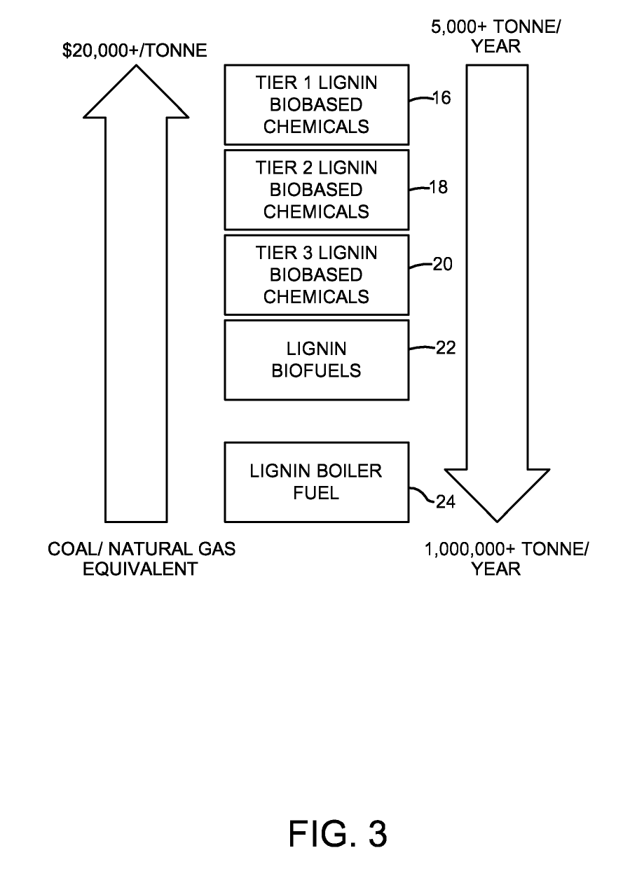

FIG. 3 provides an overview of the lignin 12 product market value and market utilization. From the illustration, market value, on a per unit product basis, may increase as market utilization may decrease. Likewise, market utilization may increase as market value may decrease. Therefore, using lignin 12 to produce the tiered biobased lignin chemicals may provide an opportunity to obtain a higher market value return for it.

In FIG. 3, the burning of lignin boiler fuel 24 by the pulp and paper mills 8 and/or cellulosic ethanol refineries 10 can represent a low market potential value in the utilization of the lignin 12. For example, the use of lignin 12 to create lignin boiler fuel 24 may provide about the same market value as using coal, natural gas, and/or other similarly-priced sources for boiler fuel. FIG. 3 also shows that the production of biobased chemicals and/or lignin biofuels 22 may offer an opportunity to extract a greater value from the lignin 12 rather than using it as lignin boiler fuel 24. The product of lignin 12 may have an economic value higher than that of boiler fuel. The processing of the lignin 12 may also produce at least two products of differing economic value. Using the tiered production strategies as shown in FIG. 3 may compound the market value captured from lignin 12. For instance, the production of tier 1 lignin biobased chemicals 16, tier 2 lignin biobased chemicals 18, and/or tier 3 lignin biobased chemicals 20 may provide a greater market value from the lignin 12. Additionally, creating lignin biofuels 22 may also provide a greater market value than lignin boiler fuel 24. From this figure, tier 1 lignin biobased chemicals 16 can provide the highest market value while tier 2 lignin biobased chemicals 18 may be less. Further, tier 3 lignin biobased chemicals 20 may be even less than tier 2 lignin biobased chemicals 18. Moreover, using lignin 12 to create these tiered biobased chemicals may still provide increased market value over using the lignin 12 as lignin boiler fuel 24.

Besides the production of biobased chemicals and/or biofuels, lignin residues from the biobased chemical and/or biofuel production can be consumed as lignin boiler fuel 24, providing for maximal use of the lignin 12 natural resource.

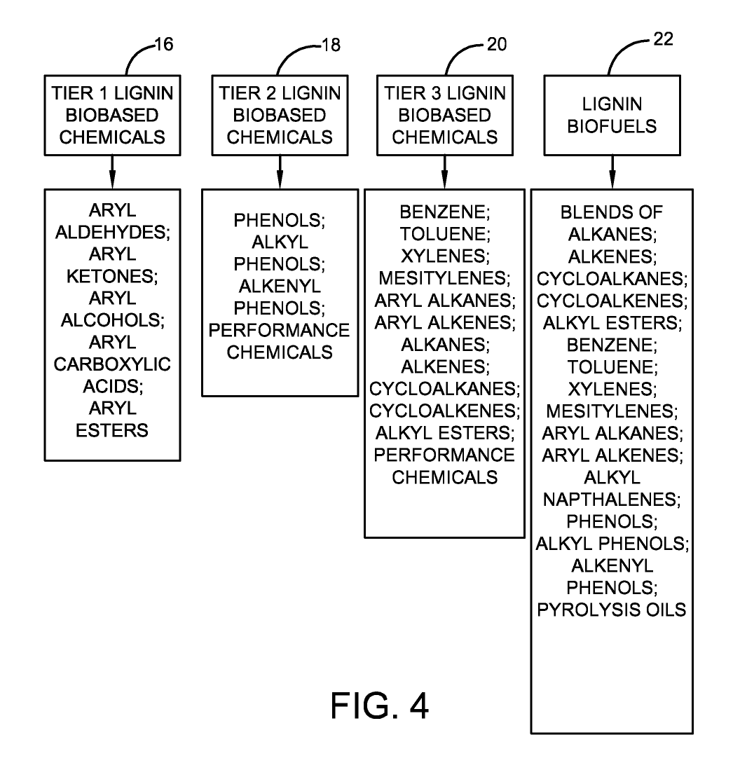

FIG. 4 provides a detailed listing of various chemicals derived from the conversion of lignin 12 to tier 1 lignin biobased chemicals 16, tier 2 lignin biobased chemicals 18, tier 3 lignin biobased chemicals 20, and/or lignin biofuels 22.

For tier 1 lignin biobased chemicals 16, lignin 12 may provide at least one chemical of aryl aldehydes, aryl carboxylic acids, aryl esters, aryl ketones, and aryl alcohols. Further, specific chemicals may include 4-hydroxybenzaldehye, vanillin, syringaldehyde, 4-hydroxybenzoic acid, vanillic acid, syringic acid, (4-hydroxyphenyl)acetaldehyde, (4-hydroxy-3-methoxyphenyl)acetaldehyde, (4-hydroxy-3,5-dimethoxyphenyl)acetaldehyde, 3-(4-hydroxyphenyl)propionaldehyde, 3-(4-hydroxy-3-methoxyphenyl)propionaldehyde, 3-(4-hydroxy-3,5-dimethoxyphenyl)propionaldehyde, 4-hydroxycinnaminaldehyde, 4-hydroxy-3-methoxycinnaminaldehyde, 4-hydroxy-3,5-dimethoxycinnaminaldehyde, (4-hydroxyphenyl)acetic acid, homovanillic acid, homosyringic acid, 3-(4-hydroxyphenyl)propionic acid, 3-(4-hydroxy-3-methoxyphenyl)propionic acid, 3-(4-hydroxy-3,5-dimethoxyphenyl)propionic acid, 4-hydroxycinnamic acid, 4-hydroxy-3-methoxycinnamic acid, and 4-hydroxy-3,5-dimethoxycinnamic acid. Aryl esters comprising a C₁-C₁₆ ester may include at least one chemical of 4-hydroxybenzoic acid, vanillic acid, syringic acid, (4-hydroxyphenyl)acetic acid, homovanillic acid, homosyringic acid, 3-(4-hydroxyphenyl)propionic acid, 3-(4-hydroxy-3-methoxyphenyl)propionic acid, 3-(4-hydroxy-3,5-dimethoxyphenyl)propionic acid, 4-hydroxycinnamic acid, 4-hydroxy-3-methoxycinnamic acid, and 4-hydroxy-3,5-dimethoxycinnamic acid. Some other tier 1 lignin biobased chemicals 16 may also include aryl ketones and aryl alcohols comprising at least one chemical of 1-(4-hydroxyphenyl)ethanone, 1-(4-hydroxy-3-methoxyphenyl)ethanone, 1-(4-hydroxy-3,5-dimethoxyphenyl)ethanone, 2-hydroxy-1-(4-hydroxyphenyl)ethanone, 2-hydroxy-1-(4-hydroxy-3-methoxyphenyl)ethanone, 2-hydroxy-1-(4-hydroxy-3,5-dimethoxyphenyl)ethanone, 1-(4-hydroxyphenyl)propanone, 1-(4-hydroxy-3-methoxyphenyl)propanone, 1-(4-hydroxy-3,5-dimethoxyphenyl)propanone, 1-(4-hydroxyphenyl)-2-methyl-1propanone, 1-(4-hydroxy-3-methoxyphenyl)-2-methyl-1-propanone, 1-(4-hydroxy-3,5-dimethoxyphenyl)-2-methyl-1-propanone, 1-(4-hydroxyphenyl)-2-propanone, 1-(4-hydroxy-3-methoxyphenyl)-2-propanone, 1-(4-hydroxy-3,5-dimethoxyphenyl)-2-propanone, 4-hydroxybenzyl alcohol, 4-hydroxy-3-methoxybenzyl alcohol, 4-hydroxy-3,5-dimethoxybenzyl alcohol, 2-(4-hydroxyphenyl)ethanol, 2-(4-hydroxy-3-methoxyphenyl)ethanol, 2-(4-hydroxy-3,5-dimethoxyphenyl)ethanol, 1-(4-hydroxyphenyl)ethanol, 1-(4-hydroxy-3-methoxyphenyl)ethanol, 1-(4-hydroxy-3,5-dimethoxyphenyl)ethanol, 1-(4-hydroxyphenyl)ethan-1,2-diol, 1-(4-hydroxy-3-methoxyphenyl)ethan-1,2-diol, 1-(4-hydroxy-3,5-dimethoxyphenyl)ethan-1,2-diol, 1-(4-hydroxyphenyl)propanol, 1-(4-hydroxy-3-methoxyphenyl)propanol, 1-(4-hydroxy-3,5-dimethoxyphenyl)propanol, 1-(4-hydroxyphenyl)propan-2-ol, 1-(4-hydroxy-3-methoxyphenyl)propan-2-ol, 1-(4-hydroxy-3,5-dimethoxyphenyl)propan-2-ol, 3-(4-hydroxyphenyl)propan-1-ol, 3-(4-hydroxy-3-methoxyphenyl)propan-1-ol, 3-(4-hydroxy-3,5-dimethoxyphenyl)propan-1-ol, 1-(4-hydroxyphenyl)propan-1,2-diol, 1-(4-hydroxy-3-methoxyphenyl)propan-1,2-diol, 1-(4-hydroxy-3,5-dimethoxyphenyl)propan-1,2-diol, 1-(4-hydroxyphenyl)propan-1,3-diol, 1-(4-hydroxy-3-methoxyphenyl)propan-1,3-diol, 1-(4-hydroxy-3,5-dimethoxyphenyl)propan-1,3-diol, 3-(4-hydroxyphenyl)propan-1,2-diol, 3-(4-hydroxy-3-methoxyphenyl)propan-1,2-diol, 3-(4-hydroxy-3,5-dimethoxyphenyl)propan-1,2-diol, 1-(4-hydroxyphenyl)propan-1,2,3-triol, 1-(4-hydroxy-3-methoxyphenyl)propan-1,2,3-triol, and 1-(4-hydroxy-3,5-dimethoxyphenyl)propan-1,2,3-triol. Moreover, some tier 1 lignin biobased chemicals 16 may include at least one chemical from aliphatic carboxylic acids comprised of formic acid, oxalic acid, acetic acid, glycolic acid, glyoxylic acid, propionic acid, lactic acid, and malonic acid.

For tier 2 lignin biobased chemicals 18, lignin 12 may provide at least one chemical of phenols, alkyl phenols, alkenyl phenols, and performance chemicals. Additionally, specific chemicals may include phenol, guaiacol, 2,6-dimethoxyphenol, 4-methylphenol, 3-methylphenol, 2-methylphenol, 4-ethylphenol, 3-ethylphenol, 2-ethylphenol, 4-propylphenol, 3-propylphenol, 2-propylphenol, 4-isopropylphenol, 3-isopropylphenol, 2-isopropylphenol, 4-butylphenol, 3-butylphenol, 2-butylphenol, 4-isobutylphenol, 3-isobutylphenol, 2-isobutylphenol, 4-t-butylphenol, 3-t-butylphenol, 2-t-butylphenol, 2,3-dimethylphenol, 2,4-dimethylphenol, 2,5-dimethylphenol, 2,6-dimethylphenol, 2,3,4-trimethylphenol, 2,4,5-trimethylphenol, 2,4,6-trimethylphenol, 2-methoxy-4-methylphenol, 2-methoxy-4-ethylphenol, 2-methoxy-4-propylphenol, 2-methoxy-4-isopropylphenol, 2-methoxy-4-butylphenol, 2-methoxy-4-isobutylphenol, 2-methoxy-4-t-butylphenol, 2,6-dimethoxy-4-methylphenol, 2,6-dimethoxy-4-ethylphenol, 2,6-dimethoxy-4-propylphenol, 2,6-dimethoxy-4-isopropylphenol, 2,6-dimethoxy-4-butylphenol, 2,6-dimethoxy-4-isobutylphenol, 2,6-dimethoxy-4-t-butylphenol, 4-hydroxystyrene, 3-methoxy-4-hydroxystyrene, 3,5-dimethoxy-4-hydroxystyrene, (4-hydroxyphenyl)-1-propene, (4-hydroxyphenyl)-2-propene, eugenol, iso-eugenol, syringeugenol, and iso-syringeugenol.

Tier 2 lignin biobased chemicals 18 may also include alkyl phenols comprising at least one of general molecular structure:

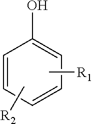

wherein R₁ is selected from among methyl, ethyl, propyl, isopropyl, butyl, isobutyl, and t-butyl; and
wherein R₂ is selected from among ethyl, propyl, isopropyl, butyl, isobutyl, and t-butyl; and
wherein R₁ and R₂ are located at positions 2, 3, 4, or 5 of the phenol ring.

Tier 2 lignin biobased chemicals 18 may also include alkyl phenols comprising at least one of general molecular structure:

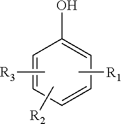

wherein R₁ and R₂ are selected from among methyl, ethyl, propyl, isopropyl, butyl, isobutyl, and t-butyl; and
wherein R₃ is selected from among ethyl, propyl, isopropyl, butyl, isobutyl, and t-butyl; and
wherein R₁, R₂, and R₃ are located at positions 2, 3, 4, or 5 of the phenol ring.

For tier 3 lignin biobased chemicals 20, lignin 12 may provide at least one chemical of benzene, toluene, xylenes, mesitylenes, aryl alkanes, aryl alkenes, alkanes, alkenes, cycloalkanes, cycloalkanes, alkyl esters, performance chemicals, and pyrolysis oils. Also, specific chemicals may include benzene, toluene, 1,2-dimethylbenzene, 1,3-dimethylbenzene, 1,4-dimethylbenzene, 1,2,3-trimethylbenzene, 1,2,4-trimethylbenzene, 1,3,5-trimethylbenzene, ethylbenzene, propylbenzene, isopropylbenzene, butylbenzene, isobutylbenzene, t-butylbenzene, styrene, 1-phenyl-1-propene, 1-phenyl-2-propene, 1-(2-methylphenyl)-1-ethene, 1-(3-methylphenyl)-1-ethene, 1-(4-methylphenyl)-1-ethene, 1-(2-methylphenyl)-1-propene, 1-(3-methylphenyl)-1-propene, 1-(4-methylphenyl)-1-propene, 1-(2-methylphenyl)-2-propene, 1-(3-methylphenyl)-2-propene, 1-(4-methylphenyl)-2-propene, hexane, heptane, octane, nonane, 2,3-dimethylheptane, 2,4-dimethylheptane, 2,3,4-trimethylheptane, 2-methyloctane, 3-methyloctane, 4-methyloctane, 2,3-dimethyloctane, 2,4-dimethyloctane, 3,4-dimethyloctane, 2,3,4-trimethyloctane, 2-methylnonane, 3-methylnonane, 4-methylnonane, 5-methylnonane, 2,3-dimethylnonane, 2,4-dimethylnonane, 2,5-dimethylnonane, 3,4-dimethylnonane, 3,5-dimethylnonane, 2,3,4-trimethylnonane, 2,4,5-trimethylnonane, 3,4,5-trimethylnonane, cyclopentane, cyclohexane, cycloheptane, methylcyclopentane, methylcyclohexane, methylcycloheptane, ethylcyclopentane, ethylcyclohexane, ethylcycloheptane, propylcyclopentane, propylcyclohexane, propylcycloheptane, isopropylcyclopentane, isopropylcyclohexane, isopropylcycloheptane, 1,2-dimethylcyclopentane, 1,3-dimethylcyclopentane, 1,2-dimethylcyclohexane, 1,3-dimethylcyclohexane, 1,4-dimethylcyclohexane, 1,2-dimethylcycloheptane, 1,3-dimethylcycloheptane, and 1,4-dimethylcycloheptane.

For the aryl alkanes, at least one chemical of a general molecular structure may comprise:

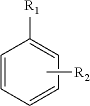

wherein R₁ is selected from among methyl, ethyl, propyl, isopropyl, butyl, isobutyl, and t-butyl; and
wherein R₂ is selected from among ethyl, propyl, isopropyl, butyl, isobutyl, and t-butyl; and
wherein R₂ is located at positions 2, 3, 4, or 5 of the ring.

For the aryl alkanes, at least one chemical of a general molecular structure may comprise:

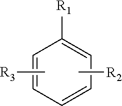

wherein R₁ and R₂ are selected from among methyl, ethyl, propyl, isopropyl, butyl, isobutyl, and t-butyl; and
wherein R₃ is selected from among ethyl, propyl, isopropyl, butyl, isobutyl, and butyl; and
wherein R₂ and R₃ are located at positions 2, 3, 4, or 5 of the ring.

For tier 3 lignin biobased chemicals 20, lignin 12 may also provide at least one chemical of alkenes comprising at least one partially unsaturated alkane. Lignin 12 may also provide at least one chemical of cycloalkenes comprising at least one partially unsaturated cycloalkane. Additionally, performance chemicals may comprise at least one chemical of benzene, toluene, xylenes, mesitylenes, aryl alkanes, aryl alkenes, alkanes, alkenes, cycloalkanes, cycloalkenes, and alkyl esters.

For the cycloalkanes, at least one chemical of a general molecular structure may comprise:

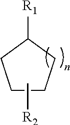

wherein n is 1, 2, or 3; and
wherein R₁ is selected from among methyl, ethyl, propyl, isopropyl, butyl, isobutyl, and t-butyl; and
wherein R₂ is selected from among ethyl, propyl, propyl, isopropyl, butyl, isobutyl, and t-butyl; and
wherein R₂ is located at any ring position other than that of R₁.

For the alkyl esters, at least on chemical of a general molecular structure may comprise:

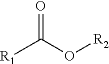

wherein R₁ and R₂ are selected from among methyl, ethyl, propyl, isopropyl, butyl, isobutyl, and t-butyl.

For lignin biofuels 22, lignin 12 may provide at least one chemical of alkanes, alkenes, cycloalkanes, cycloalkenes, alkyl esters, benzene, toluene, xylenes, mesitylenes, aryl alkanes, aryl alkenes, alkyl naphthalenes, phenols, alkyl phenols, alkenyl phenols, pyrolysis oils, and/or blends of these chemicals listed. As well, some blends of these chemicals listed as lignin biofuels 22 may comprise product mixtures of biobased chemicals of similar boiling point range. Further, the blends of the lignin biofuels 22 may comprise product mixtures of biobased chemicals with a carbon and hydrogen content of 80-100%. Also, blends of the lignin biofuels 22 may comprise product mixtures of biobased chemicals with research octane number of at least about 90.

For the lignin biofuels 22, the blends of lignin biofuels 22 can be provided for transportation fuels, heating fuels, and/or fuel additives. The transportation fuels can serve at least one market of automobile fuels, truck fuels, ship fuels, and aircraft fuels. The heating fuels may serve at least one market of home heating fuels, commercial heating fuels, and industrial boiler fuels. The fuel additives can serve at least one market of transportation fuels and heating fuels.

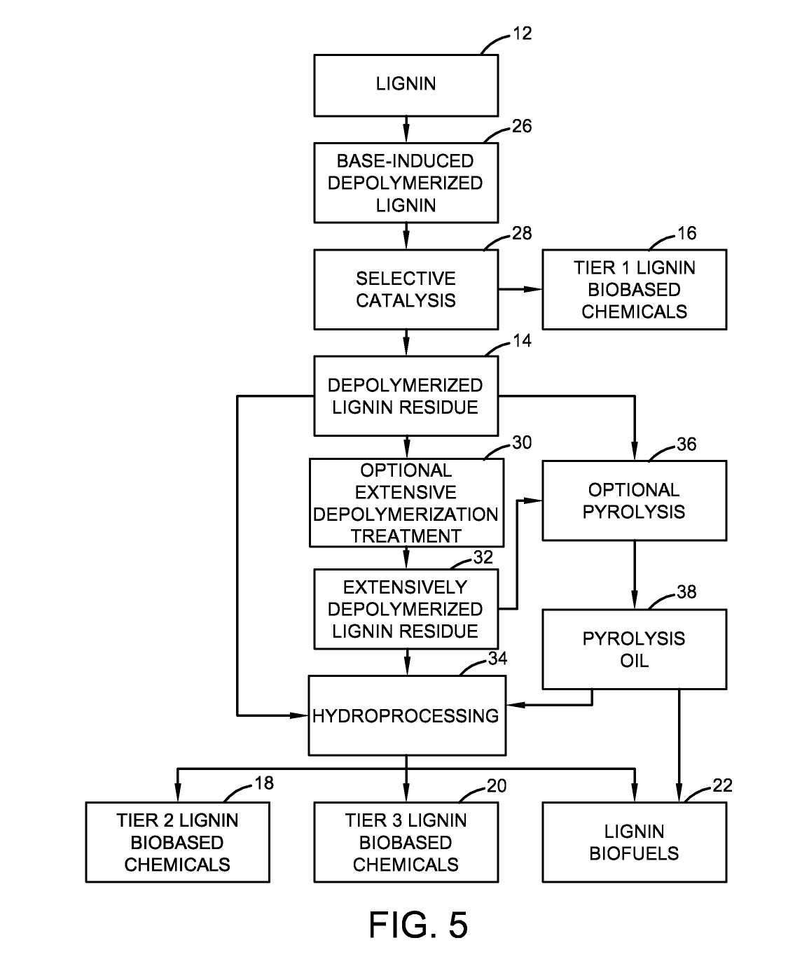

FIG. 5 details the process described in FIG. 2 in which lignin 12 undergoes depolymerization to produce tier 1 lignin biobased chemicals 16, tier 2 lignin biobased chemicals 18, tier 3 lignin biobased chemicals 20, and/or lignin biofuels 22. Within the process described in FIG. 5, several different methods may be used to provide the tiered end-products.

First, lignin 12 may be reacted under selective, mild reaction conditions to depolymerize, therefore breaking down and solubilizing the lignin 12. This process, which may be referred to as “base-induced depolymerization,” may provide base-induced depolymerized lignin 26. This based-induced depolymerized lignin 26 may be formed in situ by reaction of lignin with caustic at moderate temperatures of about 70° C. to about 250° C., more preferably at a temperature of 100° C. to about 200° C., for a period of about 15 minutes to about 120 minutes. A caustic may include, but is not limited to, sodium hydroxide, and/or potassium hydroxide, and/or calcium hydroxide solutions. The caustic solution may be aqueous, alcoholic, or mixed aqueous/alcohol. Alcohols employed in the caustic solution may include individual or mixtures thereof of C(1) to C(6) aliphatic alcohols; and most preferably methanol, ethanol, propanol, isopropanol, and/or mixtures thereof. Additionally, a co-solvent may be added to the caustic solution. When the base-induced depolymerized lignin 26 may undergo a selective catalysis 28, the production of tier 1 lignin biobased chemicals 16 can occur. For the selective catalysis 28, catalysts may include metal species comprised of, but not limited to, salts and complexes of vanadium, niobium, chromium, molybdenum, tungsten, manganese, rhenium, iron, ruthenium, osmium, cobalt, rhodium, iridium, nickel, palladium, platinum, copper, or combinations thereof. The selective catalysis 28 can allow for the production of specific chemicals within the tier 1 lignin biobased chemicals 16 to be produced.

The base-induced depolymerization and selective catalysis 28 may be completed within a vessel at about the same time. The base-induced depolymerization and selective catalysis 28 may also occur in different vessels. The base-induced depolymerization and selective catalysis 28 may also be performed sequentially.

Besides tier 1 lignin biobased chemicals 16, a caustic solution of depolymerized lignin residue 14 may be produced after selective catalysis 28. The depolymerized lignin residue 14 may comprise at least one product of biobased aromatic chemicals and biobased aromatic fuels, unreacted and/or partially reacted portions of lignin 12, and lignin boiler fuel 24. The depolymerized lignin residue 14 is further processed from at least one process of chemical processing, catalytic processing, pyrolytic processing, and biological processing. The depolymerized lignin residue 14 may be processed from at least one process of batch processing or flow processing. The depolymerized lignin residue 14 may be subjected to certain treatments, including but not limited to: 1) hydroprocessing 34, 2) an optional extensive depolymerization treatment 30, and/or 3) an optional pyrolysis 36. A further treatment of the lignin reside may include chemical-induced lignin depolymerisation, catalytic oxidation, catalytic reduction, catalytic deoxygenation, catalytic reduction/deoxygenation, and/or pyrolytic processing. After additional processing, any remaining lignin residues may provide energy production, which may be heat or power.

For the conversion of depolymerized lignin residue 14 into tier 2 lignin biobased chemicals 18, tier 3 lignin biobased chemicals 20, and/or lignin biofuels 22, the following processing options may be used, as shown in FIG. 5:
1. Hydroprocessing 34 to afford tier 2 lignin biobased chemicals 18, and/or tier 3 lignin biobased chemicals 20, and/or lignin biofuels 22.
2. An optional extensive depolymerization treatment 30 to produce an extensively depolymerized lignin residue 32, where the extensively depolymerized lignin residue 32 is then subjected to hydroprocessing 34 to afford tier 2 lignin biobased chemicals 18, and/or tier 3 lignin biobased chemicals 20, and/or lignin biofuels 22.
3. An optional pyrolysis 36 which may lead to a pyrolysis oil 38.
4. An optional extensive depolymerization treatment 30 which can be done to produce an extensively depolymerized lignin residue 32, which in turn undergoes optional pyrolysis 36. This optional pyrolysis 36 which may lead to a pyrolysis oil 38.
5. The pyrolysis oil 38 may be subjected to hydroprocessing 34 to afford tier 2 lignin biobased chemicals 18, and/or tier 3 lignin biobased chemicals 20, and/or lignin biofuels 22.
6. Alternatively, the pyrolysis oil 38 can lead directly to lignin biofuels 22 by distillation of the volatile components.

For the optional extensive depolymerization treatment 30, exposure to caustic may occur at about 200° C. to about 350° C. for about 2 hours to about 6 hours. With this exposure, an extensively depolymerized lignin residue 32 may be produced. This caustic may include, but is not limited to, sodium hydroxide, and/or potassium hydroxide, and/or calcium hydroxide solutions. The caustic solution may be aqueous, alcoholic, or mixed aqueous/alcohol. Alcohols employed in the caustic solution may include individual or mixtures thereof of C(1) to C(6) aliphatic alcohols; and most preferably methanol, ethanol, propanol, isopropanol, and/or mixtures thereof. Additionally, a co-solvent may be added to the caustic solution.

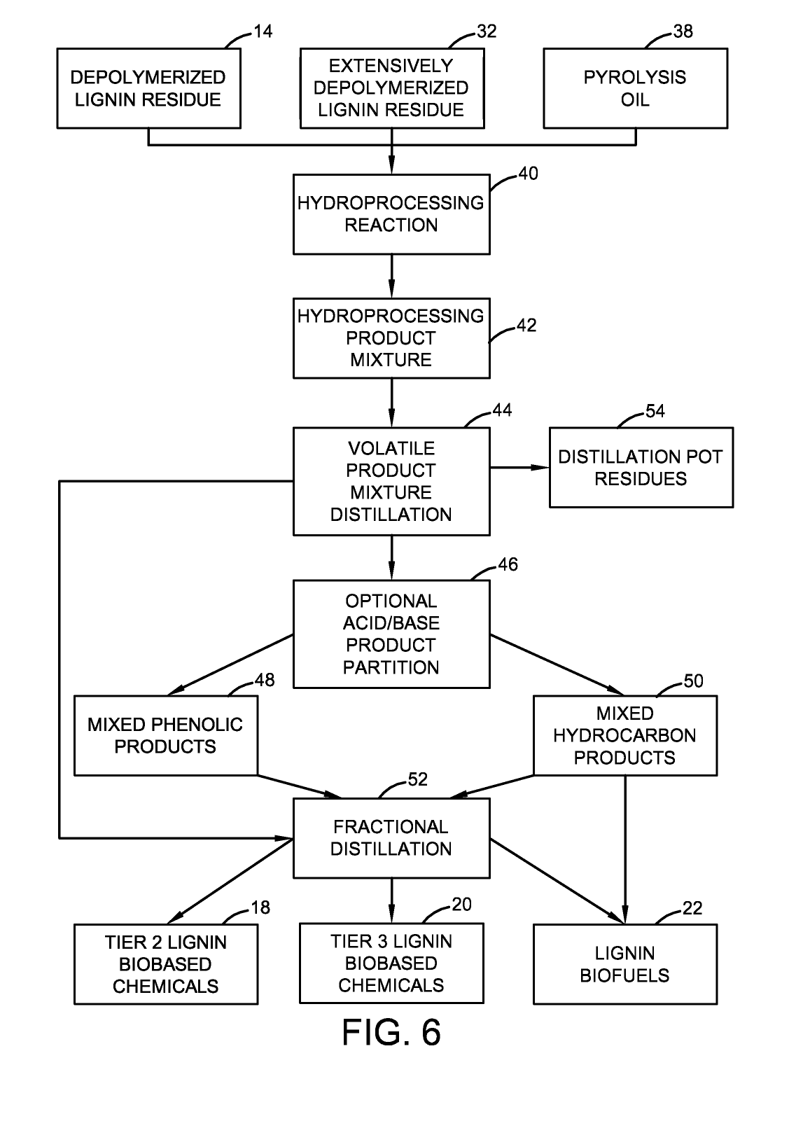

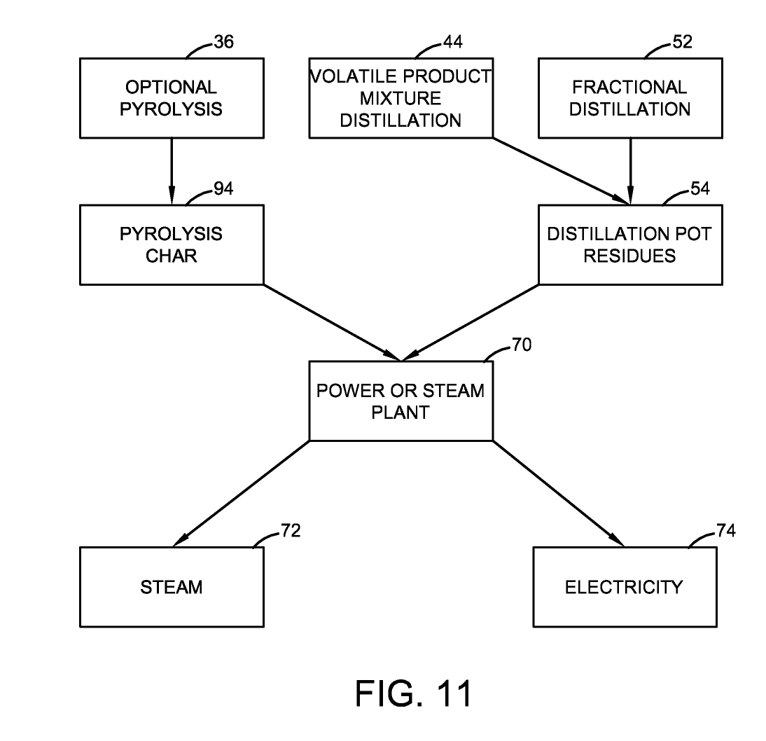

The hydroprocessing 34 method is further detailed in FIG. 6. Hydroprocessing 34 may be a multi-step method in which certain end-products may be derived from selective cracking of the lignin 12. The optional pyrolysis 36 method is further detailed in FIG. 11. The optional pyrolysis 36 may provide 1) pyrolysis oil 38 for the production of tier 2 lignin biobased chemicals 18, and/or tier 3 lignin biobased chemicals 20, and/or lignin biofuels 22; or 2) pyrolysis char 94 for the production of steam 72 and electricity 74.

FIG. 6 details the hydroprocessing 34 method. From the sources described in FIG. 5, depolymerized lignin residue 14, extensively depolymerized lignin residue 32, and/or pyrolysis oil 38 may be used. The hydroprocessing method 34 can provide a way to process these sources into tier 2 lignin biobased chemicals 18, and/or tier 3 lignin biobased chemicals 20, and/or lignin biofuels 22.

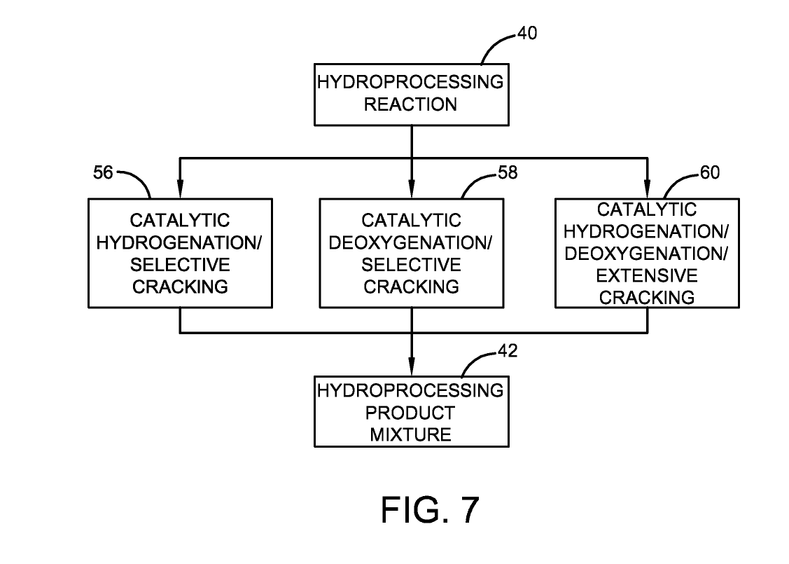

For the hydroprocessing reaction 40, a catalytic reaction may be used. Depending on the preferred tier 2 lignin biobased chemicals 18, and/or tier 3 lignin biobased chemicals 20, and/or lignin biofuels 22 end product(s), the type of catalytic reaction may differ. These catalytic reactions within the hydroprocessing reaction 40, including catalytic hydrogenation/selective cracking 56, catalytic deoxygenation/selective cracking 58, and/or catalytic hydrogenation/deoxygenation/extensive cracking 60, are detailed further in FIG. 7. A selective hydrocracking reaction wherein deoxygenation is minimized can be performed when tier 2 lignin biobased chemicals 18 may be the desired major products. An extensive hydrocracking/deoxygenation may be preferred when tier 3 lignin biobased chemicals 20 and/or lignin biofuels are the targeted major products. The hydroprocessing reaction 40 can be performed as either a single stage or dual stage process. In a dual stage process, selective hydrocracking may be conducted initially, and may be followed by an extensive hydrocracking/deoxygenation of the product stream from the first stage process.

After the hydroprocessing reaction 40, a hydroprocessing product mixture 42 may be formed. The extent to which the hydroprocessing reaction 40 takes place may impact the product mixture obtained in the hydroprocessing product mixture 42.

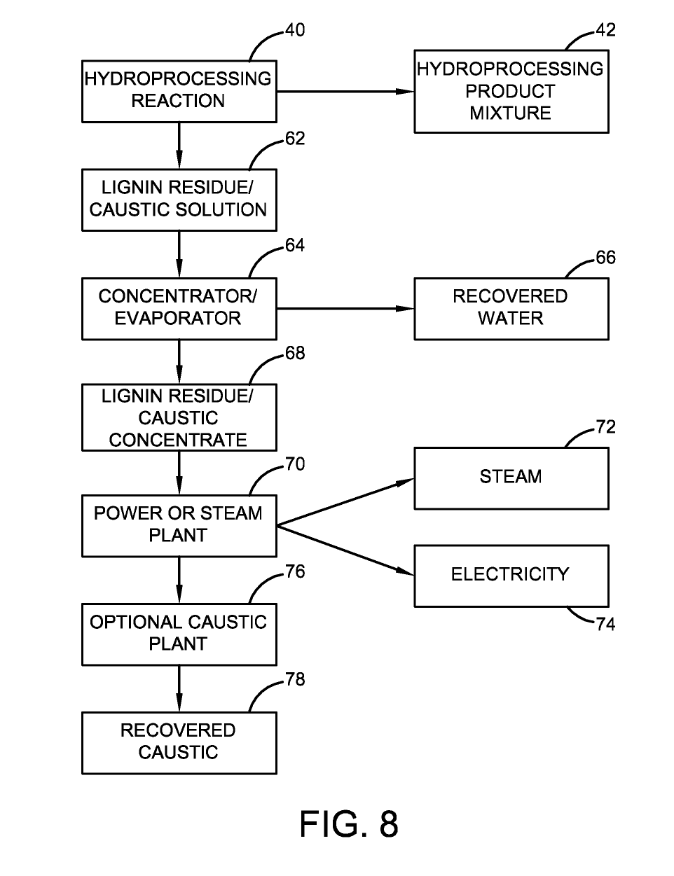

The hydroprocessing product mixture 42 can be a complex mixture of compounds arising from selective hydrocracking or extensive hydrocracking/deoxygenation of depolymerized lignin residue 14, extensively depolymerized lignin residue 32, and/or pyrolysis oil 38. A by-product of the hydroprocessing reaction 40 with a depolymerized lignin residue 14 and/or extensively depolymerized lignin residue 32 may be a lignin residue/caustic solution 62 that is described in FIG. 8. This lignin residue/caustic solution 62 may be subsequently burnt as lignin boiler fuel 24 for caustic recovery. Therefore, the caustic may be recovered and recycled in the process described herein. FIG. 8 depicts the generation of recovered water 66 when the caustic solution is of aqueous origin. When the caustic solution contains alcohol, the caustic recovery process can also generate recovered alcohol.

Returning attention now to FIG. 6, after the hydroprocessing product mixture 42 may be formed, separation of the volatile and non-volatile components of the hydroprocessing product mixture 42 may take place in a volatile product mixture distillation 44 step. Conditions for distillation of such chemicals are well documented in the literature and serve as the basis for purification of many of the same chemicals from petroleum today. The volatile product mixture from the volatile product mixture distillation 44 may then advance toward lignin biobased chemicals and biofuels.

The non-volatile component of the hydroprocessing product mixture 42 may reside in the distillation pot residues 54. The distillation pot residues 54 can then be moved to the power or steam plant 70 to be used as steam 72 and/or electricity 74, as shown in FIG. 11.

The volatile product mixture obtained from the volatile product mixture distillation 44 can then:
1. move to fractional distillation 52 for production of tier 2 lignin biobased chemicals 18, and/or tier 3 lignin biobased chemicals 20, and/or lignin biofuels 22, or
2. move to an optional acid/base product partition 46 that separates the neutral mixed hydrocarbon products 50 from the acidic mixed phenolic products 48 before proceeding to the fractional distillation 52.

After the optional acid/base product partition 46, the separated mixed phenolic products 48 can then move to a fractional distillation 52 to produce the tier 2 lignin biobased chemicals 18. Tier 2 lignin biobased chemicals 18 from the mixed phenolic products 48 can be converted to tier 3 lignin biobased chemicals 20 and/or lignin biofuels 22 by a return to hydroprocessing reaction 40. The difference between tier 2 lignin biobased chemicals 18, tier 3 lignin biobased chemicals 20, and lignin biofuels 22 may be the degree of deoxygenation. The difference between tier 3 lignin biobased chemicals 20 and lignin biofuels 22 may be that tier 3 lignin biobased chemicals 20 can be individual chemicals, or performance products thereof, whereas lignin biofuels 22 may be a blend of different chemicals of desired boiling point range and/or research octane number, and/or carbon and hydrogen content.

After the optional acid/base product partition 46, the separated mixed hydrocarbon products 50 can transfer to lignin biofuels 22, or move to fractional distillation 52 to afford tier 3 lignin biobased chemicals 20 and/or lignin biofuels 22.

FIG. 7 details the catalytic pathways within the hydroprocessing reaction 40. For the hydroprocessing reaction 40, there can be three potential methods of hydroprocessing: 1) catalytic hydrogenation/selective cracking 56, 2) catalytic deoxygenation/selective cracking 58, and/or 3) catalytic hydrogenation/deoxygenation/extensive cracking 60. These methods can be performed individually on lignin or sequentially to modify the final product distribution. These methods may also be repeated. As mentioned above in FIG. 6, the degree at which the hydroprocessing reaction 40 is conducted may impact the chemical distribution of the hydroprocessing product mixture 42.

The first of these methods for hydroprocessing is catalytic hydrogenation/selective cracking 56. The catalytic hydrogenation/selective cracking 56 method can be the mildest type of hydroprocessing reaction 40. By using the catalytic hydrogenation/selective cracking 56 method, tier 2 lignin biobased chemicals 18 may be produced. Tier 2 lignin biobased chemicals 18 may be more predominantly produced than tier 3 lignin biobased chemicals 20 and/or lignin biofuels 22.

The second method for hydroprocessing may be catalytic deoxygenation/selective cracking 58. Catalytic deoxygenation/selective cracking 58 may be a more intermediate type of reaction in that it can be harsher than catalytic hydrogenation/selective cracking 56 but milder than catalytic hydrogenation/deoxygenation/extensive cracking 60. The catalytic deoxygenation/selective cracking 58 method may lead to partial cracking and deoxygenation of the lignin 12 structure. The products of this mode of catalysis may tend to be higher molecular weight than that of catalytic hydrogenation/selective cracking 56 and catalytic hydrogenation/deoxygenation/extensive cracking 60.

The third hydroprocessing method can be catalytic hydrogenation/deoxygenation/extensive cracking 60. This method may lead to extensive cracking of the lignin 12, which may produce more tier 3 lignin biobased chemicals 20 and/or lignin biofuels 22.

FIG. 8 shows the caustic and lignin boiler fuel 24 recovery from the hydroprocessing reaction 40. The hydroprocessing reaction 40 can provide the hydroprocessing product mixture 42, as shown in FIG. 7. In addition to a hydroprocessing product mixture 42, which may be produced from the hydroprocessing reaction 40, a lignin residue/caustic solution 62 may also be formed. The separation of the lignin residue/caustic solution 62 from the hydroprocessing product mixture 42 can be achieved by any number of means including but not limited to: 1) organic solvent extraction of the organic products, 2) fractionation on ion exchange resin, and/or 3) size exclusion membranes.

The lignin residue/caustic solution 62 can then be concentrated on a concentrator/evaporator 64 to give recovered water 66 and lignin residue/caustic concentrate 68. FIG. 8 depicts the generation of recovered water 66 when the caustic solution is of aqueous origin. In the event the caustic solution contains alcohol or alcohol/water mixtures, the caustic recovery process may also generate a recovered alcohol. This reduction in water content can be considered an optional step; however, it may assist in getting complete combustion of the lignin residue when it is used in the power or steam plant 70. In any event, the recovery of alcohol from an alcoholic caustic solution before its combustion can allow for enhanced recycle of the solvent and lower raw material costs. The combustion of lignin residue/caustic concentrate 68 provides steam 72 and/or electricity 74 to the power or steam plant 70. In this regard, the spent residues of lignin from the hydroprocessing reaction 40 serve as a form of lignin boiler fuel 24. Lignin 12 combustion may be beneficial in that it may be considered to provide zero CO₂ emissions.

The pot residues of the combustion process may be sent to an optional caustic plant 76 for regeneration of recovered caustic 78. This recovered caustic 78 may be sodium hydroxide, and/or potassium hydroxide, and/or calcium hydroxide. The recovered caustic 78 may contain ash from lignin combustion. The recovered caustic 78 may be sent to further production of tiered lignin biobased chemicals and biofuels by dissolution of the combustion pot residues as appropriate into water, or alcohol, or alcohol/water mixtures and removal of the ash by filtration. The soluble alkaline filtrate can then be recycled back into yet another base-induced depolymerized lignin 26 step.

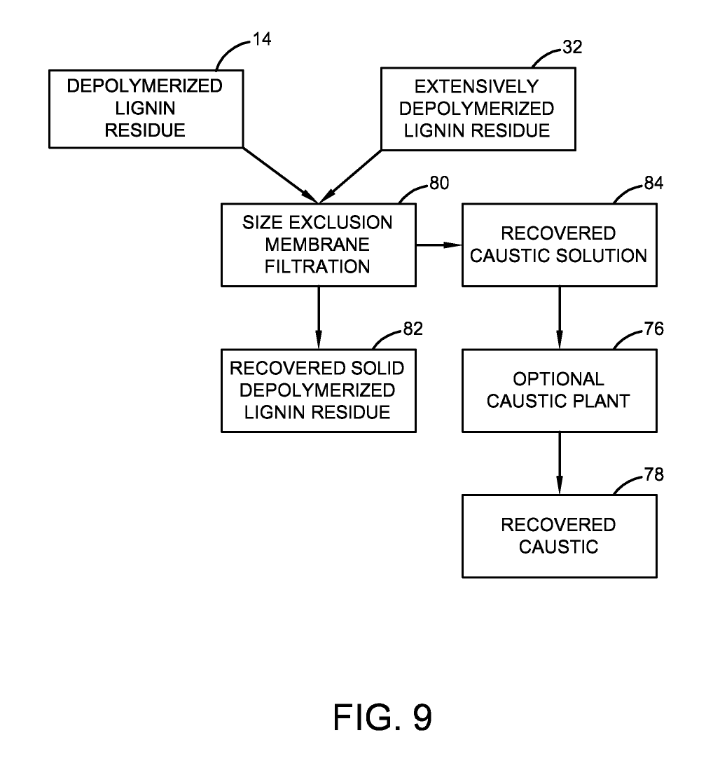

FIG. 9 provides an optional method for isolating recovered solid depolymerized lignin residue 82 for the optional pyrolysis 36 step shown in FIG. 5. Instead of using caustic treatment used in the optional extensive depolymerization treatment 30, a size exclusion membrane filtration 80 may be used to isolate the depolymerized lignin. The recovered solid depolymerized lignin residue 82 can be sent to optional pyrolysis 36, or if desired consumed as a lignin boiler fuel 24.

Depending upon the extent of tier biobased chemical and biofuel production, the lignin residue may amount from about 90% to about 10% of the original lignin 12. Most preferably, the lignin residue may amount from about 30% to about 10%, or even lower, of the origin lignin 12. Lignin residues may appear to look like lignin. However, lignin residue may be only partially reacted lignin that may be processed specifically to provide either tier 1 lignin biobased chemicals 16, and/or tier 2 lignin biobased chemicals 18, and/or tier 3 lignin biobased chemicals 20, and/or lignin biofuels 22. Lignin residue may also provide energy production.

In the process, depolymerized lignin residue 14 and/or extensively depolymerized lignin residue 32 may be subjected to size exclusion membrane filtration 80. The depolymerized lignin residue may not pass through the membrane filter, allowing for separation from the recovered caustic solution 84.

The material separated from the recovered caustic solution 84 may be recovered solid depolymerized lignin residue 82. The recovered solid depolymerized lignin residue 82 can be a metal phenolate. The recovered solid depolymerized lignin residue 82 may also be a sodium or potassium or calcium phenolate if sodium hydroxide or potassium hydroxide or calcium hydroxide, respectively, is used as the caustic in the reaction for lignin solubilization/depolymerization.

The recovered caustic solution 84 can in turn move to the optional caustic plant 76 to capture the recovered caustic 78. Like the recovered caustic 78 from the hydroprocessing reaction 40 shown in FIG. 8, the recovered caustic solution 84 in the size exclusion membrane filtration 80 option may be reused within the process, therefore recycling the caustic.

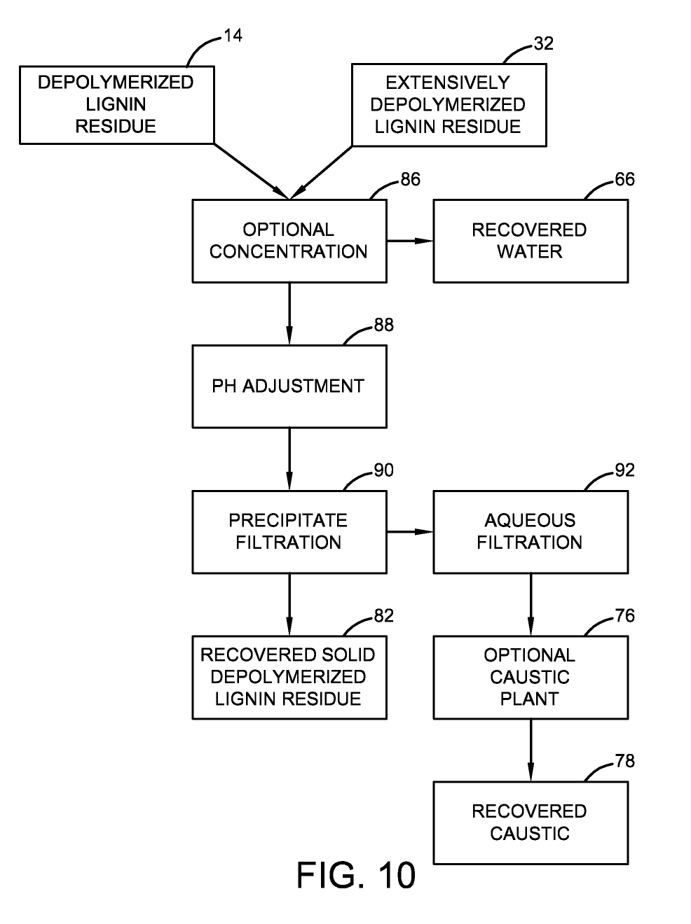

FIG. 10 provides another optional method to isolate recovered solid depolymerized lignin residue 82. As mentioned in FIG. 9, optional pyrolysis 36 of depolymerized lignin residue 14 and/or extensively depolymerized lignin residue 32 may be performed on recovered solid depolymerized lignin residue 82 from size exclusion membrane filtration 80 rather than from a caustic solution of such lignin.

FIG. 10 provides yet another optional method for providing recovered solid depolymerized lignin residue 82. In this figure, a pH precipitation may be used to provide both recovered solid depolymerized lignin residue 82 and recovered caustic 78.

With the depolymerised lignin residue 14 and/or extensively depolymerized lignin residue 32, one option to isolate the depolymerized lignin from the caustic solution can be to complete an optional concentration 86 to remove water and then complete a pH adjustment 88. The recovered water 66 may be used within the process, used within the processing facility, and/or disposed. Lowering the pH of the solution with a pH adjustment 88 step may lead to precipitation of the insoluble depolymerized lignin, also known as the recovered solid depolymerized lignin residue 82. The recovered solid depolymerized lignin residue 82 can then be separated by precipitate filtration 90. The pH adjustment 88 may take place with a mineral acid comprising, but not limited to, sulfuric acid and/or hydrochloric acid. Alternatively, organic acids such as carbon dioxide, and/or formic, and/or acetic acid can be employed in the pH adjustment 88 step. After the pH adjustment 88 using acid, a precipitate filtration 90 may separate the recovered solid depolymerized lignin residue 82. The process illustrated in FIG. 10 is for a depolymerized lignin residue 14 and/or extensively depolymerized lignin residue 32 in an aqueous solution. Alternatively the process can be applied to an alcohol or alcohol/water caustic solution of depolymerized lignin residue 14 and/or extensively depolymerized lignin residue 32. In this case, a recovered alcohol can also be generated from the optional concentration 86 step.

The recovered solid depolymerized lignin residue 82 obtained herein may be lower in metal ion content than the recovered solid depolymerized lignin residue 82 obtained in FIG. 9. However both methods outlined in FIGS. 9 and 10 may provide recovered solid depolymerized lignin residue 82 that can behave similarly in the optional pyrolysis 36 step, or serve as a lignin boiler fuel 24.

The aqueous filtrate 92 from the precipitate filtration 90 may transfer to the optional caustic plant 76 for recovery of the caustic. This recovered caustic 76 may be reused within the process, therefore recycling the caustic. The process for caustic regeneration herein may differ from that previously described in FIGS. 8 and 9 since the aqueous filtrate may contain inorganic and organic salts from acid neutralization.

FIG. 11 depicts additional ways in which certain by-products of tiered biobased chemical and biofuel production may serve as lignin boiler fuel 24 equivalents. As previously shown in FIG. 8, spent lignin residue from distillation pot residues 54 (detailed in FIG. 6) in the hydroprocessing reaction 40 may serve as a lignin boiler fuel 24 for the production of steam 72 and/or electricity 74 at a power or steam plant 70.

FIG. 11 now illustrates additional pathways that may extract maximal value from the remaining by-products. First, optional pyrolysis 36 of a depolymerized lignin may lead to pyrolysis char 94 which can be transferred as a lignin boiler fuel 24 to the power or steam plant 70. Secondly, volatile product mixture distillation 44 of the hydroprocessing product mixture 42 in FIG. 6 can yield non-volatile distillation pot residues 54 that can serve as a lignin boiler fuel 24 equivalent. Thirdly, distillation of pyrolysis oil 38 to lignin biofuels 22, as shown in FIG. 5, may lead to non-volatile distillation pot residues 54 that can also serve as a lignin boiler fuel 24 equivalent.

The embodiments have been described, hereinabove. It will be apparent to those skilled in the art that the above methods and apparatuses may incorporate changes and modifications without departing from the general scope of this invention. It is intended to include all such modifications and alterations in so far as they come within the scope of the appended claims or the equivalents thereof.

Having thus described the invention, it is now claimed:

1. A method for biorefining, comprising the steps of:
providing lignin biomass;
processing said lignin biomass; and
producing at least one product from said lignin biomass.

2. The method of claim 1, wherein said lignin biomass is provided from at least one biomass of plant biomass, woody plant biomass, agricultural plant biomass, and cultivated plant biomass.

3. The method of claim 1, wherein said lignin biomass is provided from at least one biomass of fresh plant biomass, recovered plant biomass, pulp and paper mill biomass, cellulosic ethanol refinery biomass, sugarcane mill biomass, and commercial plant biomass fractionator biomass.

4. The method of claim 1, wherein said lignin biomass is provided from kraft pulp mill lignin.

5. The method of claim 1, wherein said lignin biomass is provided from sulfite pulp mill lignin.

6. The method of claim 1, wherein said lignin biomass is provided from soda pulp mill lignin.

7. The method of claim 1, wherein said lignin biomass is provided from cellulosic ethanol refinery lignin.

8. The method of claim 1, wherein said lignin biomass is provided from commercial plant biomass fractionator lignin.

9. The method of claim 1, wherein said lignin biomass is provided from waste lignin.

10. The method of claim 9, wherein said waste lignin is provided from at least one waste lignin of recovered biomass, kraft pulp mill waste lignin, sulfite pulp mill waste lignin, soda pulp mill waste lignin, cellulosic ethanol refinery waste lignin, commercial plant biomass fractionator waste lignin, and sugar cane mill waste lignin.

11. The method of claim 1, further comprising the step of:
providing a lignin pretreatment to said lignin biomass.

12. The method of claim 1, wherein said processing of said lignin biomass is provided from at least one process of chemical processing, catalytic processing, biological processing, and pyrolytic processing.

13. The method of claim 12, wherein said processing of said lignin biomass is provided from at least one process of chemical-induced lignin depolymerization processing, catalytic oxidation processing, catalytic reduction processing, catalytic deoxygenation processing, catalytic reduction/deoxygenation processing, and pyrolytic processing.

14. The method of claim 1, wherein said processing of said lignin biomass is provided from at least one process of batch processing or flow processing.

15. The method of claim 1, further comprising the step of:
using at least one product from said lignin biomass in the tiered production of other chemicals, materials, and products.

16. The method of claim 1, wherein said at least one product from said lignin biomass comprises at least one product of biobased chemicals, biofuels, and lignin residues.

17. The method of claim 1, wherein said at least one product from said lignin biomass comprises at least two products of biobased chemicals, biofuels, and lignin residues.

18. The method of claim 16, wherein said biobased chemicals comprise at least one chemical of commodity chemicals, fine chemicals, and specialty chemicals.

19. The method of claim 16, wherein said biobased chemicals comprise at least one chemical of achiral chemicals, racemic chemicals, and chiral chemicals.

20. The method of claim 16, wherein said biobased chemicals comprise at least one chemical of aryl aldehydes, aryl carboxylic acids, aryl esters, aryl ketones, aryl alcohols, and aliphatic carboxylic acids.

21. The method of claim 16, wherein said biobased chemicals comprise at least two chemicals of aryl aldehydes, aryl carboxylic acids, aryl esters, aryl ketones, aryl alcohols, and aliphatic carboxylic acids.

22. The method of claim 20, wherein said aryl aldehydes comprise at least one chemical of 4-hydroxybenzaldehyde, vanillin, and syringaldehyde.

23. The method of claim 20, wherein said aryl aldehydes comprise at least one chemical of (4-hydroxyphenyl)acetaldehyde, (4-hydroxy-3-methoxyphenyl)acetaldehyde, (4-hydroxy-3,5-dimethoxyphenyl)acetaldehyde, 3-(4-hydroxyphenyl)propionaldehyde, 3-(4-hydroxy-3-methoxyphenyl)propionaldehyde, 3-(4-hydroxy-3,5-dimethoxyphenyl)propionaldehyde, 4-hydroxycinnaminaldehyde, 4-hydroxy-3-methoxycinnaminaldehyde, and 4-hydroxy-3,5-dimethoxycinnaminaldehyde.

24. The method of claim 20, wherein said aryl carboxylic acids comprise at least one chemical of 4-hydroxybenzoic acid, vanillic acid, and syringic acid.

25. The method of claim 20, wherein said aryl carboxylic acids comprise at least one chemical of (4-hydroxyphenyl)acetic acid, homovanillic acid, homosyringic acid, 3-(4-hydroxyphenyl)propionic acid, 3-(4-hydroxy-3-methoxyphenyl)propionic acid, 3-(4-hydroxy-3,5-dimethoxyphenyl)propionic acid, 4-hydroxycinnamic acid, 4-hydroxy-3-methoxycinnamic acid, and 4-hydroxy-3,5-dimethoxycinnamic acid.

26. The method of claim 20, wherein said aryl aldehydes and said aryl carboxylic acids comprise at least one chemical of 4-hydroxybenzaldehyde, vanillin, syringaldehyde, 4-hydroxybenzoic acid, vanillic acid, and syringic acid.

27. The method of claim 20, wherein said aryl esters comprise a C₁-C₁₆ ester of at least one chemical of 4-hydroxybenzoic acid, vanillic acid, syringic acid, (4-hydroxyphenyl)acetic acid, homovanillic acid, homosyringic acid, 3-(4-hydroxyphenyl)propionic acid, 3-(4-hydroxy-3-methoxyphenyl)propionic acid, 3-(4-hydroxy-3,5-dimethoxyphenyl)propionic acid, 4-hydroxycinnamic acid, 4-hydroxy-3-methoxycinnamic acid, and 4-hydroxy-3,5-dimethoxycinnamic acid.

28. The method of claim 20, wherein said aryl ketones comprise at least one chemical of 1-(4-hydroxyphenyl)ethanone, 1-(4-hydroxy-3-methoxyphenyl)ethanone, and 1-(4-hydroxy-3,5-dimethoxyphenyl)ethanone.

29. The method of claim 20, wherein said aryl ketones comprise at least one chemical of 2-hydroxy-1-(4-hydroxyphenyl)ethanone, 2-hydroxy-1-(4-hydroxy-3-methoxyphenyl)ethanone, 2-hydroxy-1-(4-hydroxy-3,5-dimethoxyphenyl)ethanone, 1-(4-hydroxyphenyl)propanone, 1-(4-hydroxy-3-methoxyphenyl)propanone, 1-(4-hydroxy-3,5-dimethoxyphenyl)propanone, 1-(4-hydroxyphenyl)-2-methyl-1-propanone, 1-(4-hydroxy-3-methoxyphenyl)-2-methyl-1-propanone, 1-(4-hydroxy-3,5-dimethoxyphenyl)-2-methyl-1-propanone, 1-(4-hydroxyphenyl)-2-propanone, 1-(4-hydroxy-3-methoxyphenyl)-2-propanone, and 1-(4-hydroxy-3,5-dimethoxyphenyl)-2-propanone.

30. The method of claim 20, wherein said aryl alcohols comprise at least one chemical of 4-hydroxybenzyl alcohol, 4-hydroxy-3-methoxybenzyl alcohol, 4-hydroxy-3,5-dimethoxybenzyl alcohol, 2-(4-hydroxyphenyl)ethanol, 2-(4-hydroxy-3-methoxyphenyl)ethanol, 2-(4-hydroxy-3,5-dimethoxyphenyl)ethanol, 1-(4-hydroxyphenyl)ethanol, 1-(4-hydroxy-3-methoxyphenyl)ethanol, 1-(4-hydroxy-3,5-dimethoxyphenyl)ethanol, 1-(4-hydroxyphenyl)ethan-1,2-diol, 1-(4-hydroxy-3-methoxyphenyl)ethan-1,2-diol, 1-(4-hydroxy-3,5-dimethoxyphenyl)ethan-1,2-diol, 1-(4-hydroxyphenyl)propanol, 1-(4-hydroxy-3-methoxyphenyl)propanol, 1-(4-hydroxy-3,5-dimethoxyphenyl)propanol, 1-(4-hydroxyphenyl)propan-2-ol, 1-(4-hydroxy-3-methoxyphenyl)propan-2-ol, 1-(4-hydroxy-3,5-dimethoxyphenyl)propan-2-ol, 3-(4-hydroxyphenyl)propan-1-ol, 3-(4-hydroxy-3-methoxyphenyl)propan-1-ol, 3-(4-hydroxy-3,5-dimethoxyphenyl)propan-1-ol, 1-(4-hydroxyphenyl)propan-1,2-diol, 1-(4-hydroxy-3-methoxyphenyl)propan-1,2-diol, 1-(4-hydroxy-3,5-dimethoxyphenyl)propan-1,2-diol, 1-(4-hydroxyphenyl)propan-1,3-diol, 1-(4-hydroxy-3-methoxyphenyl)propan-1,3-diol, 1-(4-hydroxy-3,5-dimethoxyphenyl)propan-1,3-diol, 3-(4-hydroxyphenyl)propan-1,2-diol, 3-(4-hydroxy-3-methoxyphenyl)propan-1,2-diol, 3-(4-hydroxy-3,5-dimethoxyphenyl)propan-1,2-diol, 1-(4-hydroxyphenyl)propan-1,2,3-triol, 1-(4-hydroxy-3-methoxyphenyl)propan-1,2,3-triol, and 1-(4-hydroxy-3,5-dimethoxyphenyl)propan-1,2,3-triol.

31. The method of claim 20, wherein said aliphatic carboxylic acids comprise at least one chemical of formic acid, oxalic acid, acetic acid, glycolic acid, glyoxylic acid, propionic acid, lactic acid, and malonic acid.

32. The method of claim 16, wherein said biobased chemicals comprise at least one chemical of phenols, alkyl phenols, alkenyl phenols, and performance chemicals.

33. The method of claim 16, wherein said biobased chemicals comprise at least two chemicals of phenols, alkyl phenols, alkenyl phenols, and performance chemicals.

34. The method of claim 32, wherein said phenols comprise at least one chemical of phenol, guaiacol, and 2,6-dimethoxyphenol.

35. The method of claim 32, wherein said alkyl phenols comprise at least one chemical of 4-methylphenol, 3-methylphenol, 2-methylphenol, 4-ethylphenol, 3-ethylphenol, 2-ethylphenol, 4-propylphenol, 3-propylphenol, 2-propylphenol, 4-isopropylphenol, 3-isopropylphenol, 2-isopropylphenol, 4-butylphenol, 3-butylphenol, 2-butylphenol, 4-isobutylphenol, 3-isobutylphenol, 2-isobutylphenol, 4-t-butylphenol, 3-t-butylphenol, 2-t-butylphenol, 2,3-dimethylphenol, 2,4-dimethylphenol, 2,5-dimethylphenol, 2,6-dimethylphenol, 2,3,4-trimethylphenol, 2,4,5-trimethylphenol, and 2,4,6-trimethylphenol.

36. The method of claim 32, wherein said alkyl phenols comprise at least one chemical of a general molecular structure: 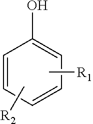
wherein R₁ is selected from among methyl, ethyl, propyl, isopropyl, butyl, isobutyl, and t-butyl; and
wherein R₂ is selected from among ethyl, propyl, isopropyl, butyl, isobutyl, and t-butyl; and
wherein R₁ and R₂ are located at positions 2, 3, 4, or 5 of the phenol ring.

37. The method of claim 32, wherein said alkyl phenols comprise at least one chemical of a general molecular structure: 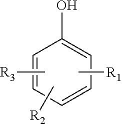
wherein R₁ and R₂ are selected from among methyl, ethyl, propyl, isopropyl, butyl, isobutyl, and t-butyl; and
wherein R₃ is selected from among ethyl, propyl, isopropyl, butyl, isobutyl, and t-butyl; and
wherein R₁, R₂, and R₃ are located at positions 2, 3, 4, or 5 of the phenol ring.

38. The method of claim 32, wherein said alkyl phenols comprise at least one chemical of 2-methoxy-4-methylphenol, 2-methoxy-4-ethylphenol, 2-methoxy-4-propylphenol, 2-methoxy-4-isopropylphenol, 2-methoxy-4-butylphenol, 2-methoxy-4-isobutylphenol, 2-methoxy-4-t-butylphenol, 2,6-dimethoxy-4-methylphenol, 2,6-dimethoxy-4-ethylphenol, 2,6-dimethoxy-4-propylphenol, 2,6-dimethoxy-4-isopropylphenol, 2,6-dimethoxy-4-butylphenol, 2,6-dimethoxy-4-isobutylphenol, and 2,6-dimethoxy-4-t-butylphenol.

39. The method of claim 32, wherein said alkenyl phenols comprise at least one chemical of 4-hydroxystyrene, 3-methoxy-4-hydroxystyrene, 3,5-dimethoxy-4-hydroxystyrene, (4-hydroxyphenyl)-1-propene, (4-hydroxyphenyl)-2-propene, eugenol, iso-eugenol, syringeugenol, and iso-syringeugenol.

40. The method of claim 32, wherein said performance chemicals comprise at least one chemical of products comprising phenols, alkyl phenols, and alkenyl phenols.

41. The method of claim 16, wherein said biobased chemicals comprise at least one chemical of benzene, toluene, xylenes, mesitylenes, aryl alkanes, aryl alkenes, alkanes, alkenes, cycloalkanes, cycloalkenes, alkyl esters, performance chemicals, and pyrolysis oils.

42. The method of claim 16, wherein said biobased chemicals comprise at least two chemicals of benzene, toluene, xylenes, mesitylenes, aryl alkanes, aryl alkenes, alkanes, alkenes, cycloalkanes, cycloalkenes, alkyl esters, performance chemicals, and pyrolysis oils.

43. The method of claim 41, wherein said biobased chemicals comprise at least one chemical of benzene, toluene, 1,2-dimethylbenzene, 1,3-dimethylbenzene, 1,4-dimethylbenzene, 1,2,3-trimethylbenzene, 1,2,4-trimethylbenzene and 1,3,5-trimethylbenzene.

44. The method of claim 41, wherein said aryl alkanes comprise at least one chemical of ethylbenzene, propylbenzene, isopropylbenzene, butylbenzene, isobutylbenzene, and t-butylbenzene.

45. The method of claim 41, wherein said aryl alkanes comprise at least one chemical of a general molecular structure: 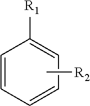
wherein R₁ is selected from among methyl, ethyl, propyl, isopropyl, butyl, isobutyl, and t-butyl; and
wherein R₂ is selected from among ethyl, propyl, isopropyl, butyl, isobutyl, and t-butyl; and
wherein R₂ is located at positions 2, 3, 4, or 5 of the ring.

46. The method of claim 41, wherein said aryl alkanes comprise at least one chemical of a general molecular structure: 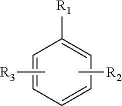
wherein R₁ and R₂ are selected from among methyl, ethyl, propyl, isopropyl, butyl, isobutyl, and t-butyl; and
wherein R₃ is selected from among ethyl, propyl, isopropyl, butyl, isobutyl, and t-butyl; and
wherein R₂ and R₃ are located at positions 2, 3, 4, or 5 of the ring.

47. The method of claim 41, wherein said aryl alkenes comprise at least one chemical of styrene, 1-phenyl-1-propene, 1-phenyl-2-propene, 1-(2-methylphenyl)-1-ethene, 1-(3-methylphenyl)-1-ethene, 1-(4-methylphenyl)-1-ethene, 1-(2-methylphenyl)-1-propene, 1-(3-methylphenyl)-1-propene, 1-(4-methylphenyl)-1-propene, 1-(2-methylphenyl)-2-propene, 1-(3-methylphenyl)-2-propene, and 1-(4-methylphenyl)-2-propene.

48. The method of claim 41, wherein said alkanes comprise at least one chemical of hexane, heptane, octane, nonane, 2,3-dimethylheptane, 2,4-dimethylheptane, 2,3,4-trimethylheptane, 2-methyloctane, 3-methyloctane, 4-methyloctane, 2,3-dimethyloctane, 2,4-dimethyloctane, 3,4-dimethyloctane, 2,3,4-trimethyloctane, 2-methylnonane, 3-methylnonane, 4-methylnonane, 5-methylnonane, 2,3-dimethylnonane, 2,4-dimethylnonane, 2,5-dimethylnonane, 3,4-dimethylnonane, 3,5-dimethylnonane, 2,3,4-trimethylnonane, 2,4,5-trimethylnonane, and 3,4,5-trimethylnonane.

49. The method of claim 41, wherein said alkenes comprise at least one compound of a partially unsaturated alkane.

50. The method of claim 41, wherein said cycloalkanes comprise at least one chemical of cyclopentane, cyclohexane, cycloheptane, methylcyclopentane, methylcyclohexane, methylcycloheptane, ethylcyclopentane, ethylcyclohexane, ethylcycloheptane, propylcyclopentane, propylcyclohexane, propylcycloheptane, isopropylcyclopentane, isopropylcyclohexane, isopropylcycloheptane, 1,2-dimethylcyclopentane, 1,3-dimethylcyclopentane, 1,2-dimethylcyclohexane, 1,3-dimethylcyclohexane, 1,4-dimethylcyclohexane, 1,2-dimethylcycloheptane, 1,3-dimethylcycloheptane, and 1,4-dimethylcycloheptane.

51. The method of claim 41, wherein said cycloalkanes comprise at least one chemical of a general molecular structure: 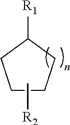
wherein n is 1, 2, or 3; and
wherein R₁ is selected from among methyl, ethyl, propyl, isopropyl, butyl, isobutyl, and t-butyl; and
wherein R₂ is selected from among ethyl, propyl, propyl, isopropyl, butyl, isobutyl, and t-butyl; and
wherein R₂ is located at any ring position other than that of R₁.

52. The method of claim 41, wherein said cycloalkenes comprise at least one compound of a partially unsaturated cycloalkane.

53. The method of claim 41, wherein said alkyl esters comprise at least one chemical of a general molecular structure: 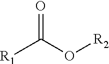
wherein R₁ and R₂ are selected from among methyl, ethyl, propyl, isopropyl, butyl, isobutyl, and t-butyl.

54. The method of claim 41, wherein said performance chemicals comprise at least one chemical of benzene, toluene, xylenes, mesitylenes, aryl alkanes, aryl alkenes, alkanes, alkenes, cycloalkanes, cycloalkenes, and alkyl esters.

55. The method of claim 16, wherein said biofuels comprise at least one biobased chemical of alkanes, alkenes, cycloalkanes, cycloalkenes, alkyl esters, benzene, toluene, xylenes, mesitylenes, aryl alkanes, aryl alkenes, alkyl naphthalenes, phenols, alkyl phenols, alkenyl phenols, and pyrolysis oils.

56. The method of claim 16, wherein said biofuels comprise blends of at least two biobased chemicals of alkanes, alkenes, cycloalkanes, cycloalkenes, alkyl esters, benzene, toluene, xylenes, mesitylenes, aryl alkanes, aryl alkenes, alkyl naphthalenes, phenols, alkyl phenols, alkenyl phenols, and pyrolysis oils.

57. The method of claim 55, wherein said blends of said biofuels comprise product mixtures of biobased chemicals of similar boiling point range.

58. The method of claim 55, wherein said blends of said biofuels comprise product mixtures of biobased chemicals with a carbon and hydrogen content of about 80% to about 100%.

59. The method of claim 55, wherein said blends of said biofuels comprise product mixtures of biobased chemicals with a research octane number of at least about 90.

60. The method of claim 55, wherein said blends of said biofuels are comprised of at least one fuel of transportation fuels, heating fuels, and fuel additives.

61. The method of claim 60 wherein said transportation fuels serve at least one market of automobile fuels, truck fuels, ship fuels, and aircraft fuels.

62. The method of claim 60, wherein said heating fuels serve at least one market of home heating fuels, commercial heating fuels, and industrial boiler fuels.

63. The method of claim 60, wherein said fuel additives serve at least one market of transportation fuels and heating fuels.

64. The method of claim 16, wherein said lignin residue is subjected to further processing to produce at least one additional product.

65. The method of claim 64, wherein said lignin residue is further processed from at least one process of chemical processing, catalytic processing, pyrolytic processing, and biological processing.

66. The method of claim 64, wherein said lignin residue is further processed from at least one process of chemical-induced lignin depolymerisation, catalytic oxidation, catalytic reduction, catalytic deoxygenation, catalytic reduction/deoxygenation, and pyrolytic processing.

67. The method of claim 64, wherein said lignin residue is further processed from at least one process of batch processing or flow processing.

68. The method of claim 16 wherein said lignin residues comprise at least one chemical of biobased aromatic chemicals and biobased aromatic fuels.

69. The method of claim 16, wherein said lignin residues provide energy production.

70. The method of claim 69, wherein said energy production is heat or power.

71. The method of claim 1, wherein said lignin biomass has a weight, and a waste product of said lignin biomass is less than 30% of said lignin biomass weight.

72. The method of claim 1, wherein said lignin biomass has a weight, and a waste product of said lignin biomass is less than 20% of said lignin biomass weight.

73. The method of claim 1, wherein said lignin biomass has a weight, and a waste product of said lignin biomass is less than 10% of said lignin biomass weight.

74. The method of claim 1, wherein waste products of said processing of said lignin biomass provide energy production.

75. The method of claim 74, wherein said energy production is heat or power.

76. The method of claim 1, further comprising the step of:
recovering and recycling caustic from said processing of said lignin biomass.

77. The method of claim 76, wherein size exclusion membrane filtration is used for said recovering and recycling caustic from said processing of said lignin biomass.

78. The method of claim 76, wherein a pH precipitation is used for said recovering and recycling caustic from said processing of said lignin biomass.

79. The method of claim 1, further comprising the step of functionalizing said lignin biomass prior to said producing at least one product from said lignin biomass.

80. The method of claim 1, wherein said product of said lignin biomass has an economic value higher than boiler fuel.

81. The method of claim 1, wherein said processing of said lignin biomass produces at least two products of differing economic value.

82. The method of claim 1, wherein selective production of said product from said lignin biomass occurs.

83. A method for biorefining, comprising the steps of:
providing lignin biomass comprising at least one biomass of woody plant biomass, agricultural plant biomass, cultivated plant biomass, kraft pulping biomass, sulfite pulping biomass, soda pulping biomass, cellulosic ethanol refinery biomass, sugarcane mill biomass, and waste biomass;
processing said lignin biomass from at least one process of chemical processing, catalytic processing, biological processing, and pyrolytic processing;
processing said lignin biomass from at least one process of chemical-induced lignin depolymerization processing, catalytic oxidation processing, catalytic reduction processing, catalytic deoxygenation processing, catalytic reduction/deoxygenation processing, and pyrolytic processing;
functionalizing said lignin biomass prior to producing at least one product from said lignin biomass;
producing at least one product from said lignin biomass comprising at least one product of biobased aromatic chemicals, biobased aromatic fuels, and lignin residues;
producing a plurality of products from said lignin biomass comprising at least one chemical of aryl aldehydes, aryl carboxylic acids, aryl esters, aryl ketones, aryl alcohols, aliphatic carboxylic acids, phenols, alkyl phenols, alkenyl phenols, performance chemicals, benzene, toluene, xylenes, mesitylenes, aryl alkanes, aryl alkenes, alkanes, alkenes, cycloalkanes, cycloalkenes, alkyl esters, performance chemicals, pyrolysis oils, alkyl naphthalenes, phenols, and alkyl phenols;
reducing the waste product of said lignin biomass, wherein said lignin biomass has a weight, and said waste product of said lignin biomass is less than 20% of said lignin biomass weight;
producing energy utilizing said lignin residues;
producing energy utilizing said waste product of said lignin biomass biomass; and
recovering and recycling caustic from said processing of said lignin;
wherein selective production of said product of said lignin biomass occurs.
